(19) United States
(12) Patent Application Publication
(10) Pub. No.: US 2025/0205306 A1
(43) Pub. Date: Jun. 26, 2025

(54) USE OF FOLLISTATIN-LIKE 1 (FSTL1) IN CARDIOMETABOLIC HEART FAILURE

(71) Applicant: Regencor, Inc., San Carlos, CA (US)

(72) Inventors: Thomas B. OKARMA, San Carlos, CA (US); Pilar RUIZ-LOZANO, San Carlos, CA (US)

(21) Appl. No.: 18/849,801

(22) PCT Filed: Mar. 24, 2023
(86) PCT No.: PCT/US2023/064950

§ 371 Date: Sep. 23, 2024

Related U.S. Application Data
(60) Provisional application No. 63/324,018, filed on Mar. 25, 2022.
(60) Provisional application No. 63/326,718, filed on Apr. 1, 2022.

(51) Int. Cl. A61K 38/17; A61K 31/7072; A61P 9/04

(52) U.S. Cl. CPC A61K 38/1709; A61K 31/7072; A61P 9/04

(57) ABSTRACT

Provided herein, inter alia, are compositions and kits comprising epicardial-derived paracrine factors, such as follistatin-like 1 (FSTL1), for aiding treatment of cardiometabolic heart failures, as well as methods for using the same.

### RELATED APPLICATIONS

This application is a U.S. National Stage Application based on International Application No. PCT/US2023/064950, filed Mar. 24, 2023, and claims priority to U.S. Provisional Patent Application Ser. Nos. 63/324,018, filed on Mar. 25, 2022, and 63/326,718, filed on Apr. 1, 2022, which are incorporated herein by reference in their entireties for all purposes.

### STATEMENT REGARDING FEDERALLY SPONSORED RESEARCH OR DEVELOPMENT

This invention was made with government support under Grant No. R44HL140649 award by National Institutes of Health, Heart and Lung Institute. The government has certain rights in the invention.

### INCORPORATION OF THE SEQUENCE LISTING

This application contains a Sequence Listing, which is hereby incorporated herein by reference in its entirety. The accompanying Sequence Listing text file, named “Sequence_Listing_ST26 048722-505001WO” was created on Mar. 8, 2023 and is 7.4 KB.

### BACKGROUND

Heart failure is a complex clinical syndrome that can result from either functional or structural impairment of ventricles resulting in symptomatic left ventricle dysfunction. The symptoms come from an inadequate cardiac output, failing to keep up with the metabolic demands of the body. It is a leading cause of cardiovascular morbidity and mortality worldwide despite the advances in therapies and prevention. It can result from disorders of the pericardium, myocardium, endocardium, heart valves, great vessels, or some metabolic abnormalities.

The cardiometabolic syndrome is a prevalent metabolic disorder. Epidemiologic studies correlate the cardiometabolic syndrome with an increased risk of coronary heart disease, ischemic stroke, cardiovascular mortality, and total mortality. There is also evidence that the cardiometabolic syndrome is a risk factor for abnormalities in myocardial metabolism, cardiac dysfunction, and arrhythmias. Diabetic cardiomyopathy is increasingly problematic clinically, with the combination of metabolic syndrome and chronic inflammation greatly increasing the risk for both coronary and peripheral artery disease.

There is a need for therapies that can address and/or treat cardiometabolic heart failure (e.g., diabetic cardiomyopathy). The present disclosed herein addresses these needs and provides additional benefits as well.

### SUMMARY

Provided herein, inter alia, is a method of aiding heart failure treatment, the method including contacting the cardiac tissue with a non-glycosylated or hypoglycosylated follistatin-like 1 (FSTL1) polypeptide, wherein the heart failure can be cardiometabolic heart failure. Also provided herein is a method of treating cardiometabolic heart failure, the method including contacting cardiac tissue with a non-glycosylated follistatin-like 1 (FSTL1) polypeptide. Further provided herein is a method of repairing cardiac tissue following cardiometabolic heart failure, the method including contacting the cardiac tissue with a non-glycosylated follistatin-like 1 (FSTL1) polypeptide.

In any of the methods described herein, the cardiometabolic heart failure can be a heart failure with reduced ejection fraction (HFrEF). In some embodiments, the cardiometabolic heart failure can be a diabetic cardiomyopathy. In some embodiments, the FSTL1 can decrease heart failure mortality. In some embodiments, the FSTL1 can attenuate the development of coronary and cerebral vascular dysfunction. In some embodiments, the FSTL1 can improve cardiac function. In some embodiments, the FSTL1 can reduce heart rate. In some embodiments, the FSTL1 does not alter left ventricular volume and systolic or diastolic volume. In some embodiments, the FSTL1 can prevent left ventricular diastolic dysfunction. In some embodiments, the FSTL1 can improve ventricular-vascular interactions. In some embodiments, the FSTL1 can decrease pulmonary congestion. In some embodiments, the FSTL1 can prevent mismatch between ventricular function and arterial load as a function of decreased contractility. In some embodiments, the FSTL1 can prevent the narrowing of pulse pressure. In some embodiments, the FSTL1 can improve coronary and/or peripheral vascular function. In some embodiments, the FSTL1 can increase blood flow to the heart and skeletal muscles. In some embodiments, the FSTL1 can attenuate impaired BK_{Ca} channel-mediated coronary arteriole dilatory capacity in remote coronary vessels. In some embodiments, the FSTL1 can attenuate the loss of TXA₂-mediated vasoconstriction. In some embodiments, the FSTL1 can improve mitochondrial function. In some embodiments, the FSTL1 can reduce necrosis in infarct area. In some embodiments, the FSTL1 can reduce lung weight.

In some embodiments, the FSTL1 can be delivered by systemically. In some embodiments, the FSTL1 can be delivered endocardially. In some embodiments, the endocardial delivery can be via a catheter. In some embodiments, the FSTL1 can be delivered epicardially. In some embodiments, the cardiac tissue can be contacted from one or more of an epicardial site, an endocardial site, and/or through direct injection into the myocardium. In some embodiments, the FSTL1 can be delivered using a drug-eluting stent. In some embodiments, the FSTL1 can be delivered by a hydrogel embedded or seeded with the FSTL1. In some embodiments, the FSTL1 can be delivered by a collagen patch embedded or seeded with the FSTL1. In some embodiments, the FSTL1 can be delivered via an osmotic pump. In some embodiments, the FSTL1 can be delivered as a single or several subcutaneous bolus. In some embodiments, the FSTL1 can be delivered by a coronary infusion.

In some embodiments, the FSTL1 can be expressed in the heart by use of modified RNAs (modRNAs). In some embodiments, the FSTL1 can be expressed by genomic editing.

In some embodiments, the method further comprising an inhibitor of FSTL1 glycosylation. In some embodiments, the inhibitor of FSTL1 glycosylation comprises tunicamycin.

Each of the aspects and embodiments described herein are capable of being used together, unless excluded either explicitly or clearly from the context of the embodiment or aspect.

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic summary of an experimental design of Example 1. Intact female Ossabaw swine (2 mo. old) were fed a Western Diet for 4 months to develop metabolic syndrome. At 6 months of age, animals were subjected to 90 minutes ischemia followed by reperfusion (I/R) to induce MI. One month post-MI, ALZET osmotic pumps were implanted and either vehicle (MI group) or FSTL1 (MI+FSTL1 group) was delivered over two weeks, with terminal in vitro vascular experiments performed 2 months post MI.

FIGS. 2A-2B collectively illustrate the effect of FSTL1 treatment on insulin resistance and systolic function. Heart failure (HF) group and HF group treated with FSTL1 (HF+FSTL1).

FIG. 2A is a graph illustrating the effect of FSTL1 treatment on insulin resistance.

FIG. 2B is a graph illustrating the effect of FSTL1 treatment on ejection fraction.

FIG. 3 is a graph illustrating decreased mortality following treatment with FSTL1.

FIGS. 4A-4B collectively illustrate infarct size differences between heart failure (HF) group and HF group treated with FSTL1 (HF+FSTL1).

FIG. 4A is a graph illustrating relative infarct size between heart failure (HF) group and HF group treated with FSTL1 (HF+FSTL1).

FIG. 4B illustrates infarct size differences between heart failure (HF) group and HF group treated with FSTL1 (HF+FSTL1).

FIGS. 5A-5B collectively illustrate that heart rate can be reduced following FSTL1 treatment. Heart failure (HF) group and HF group treated with FSTL1 (HF+FSTL1).

FIG. 5A is a graph illustrating the effect of FSTL1 treatment on heart rate.

FIG. 5B is a graph illustrating the effect of FSTL1 treatment on left ventricular stroke volume.

FIG. 6 is a graph illustrating that FSTL1 treatment does not alter left ventricular end systolic or diastolic volume. Heart failure (HF) group and HF group treated with FSTL1 (HF+FSTL1).

FIGS. 7A-7C collectively illustrate that FSTL1 prevents left ventricular diastolic dysfunction. Heart failure (HF) group and HF group treated with FSTL1 (HF+FSTL1).

FIG. 7A is a graph illustrating the effect of FSTL1 treatment on left ventricular end systolic pressure.

FIG. 7B is a graph illustrating the effect of FSTL1 treatment on left ventricular end diastolic pressure.

FIG. 7C is a graph illustrating the effect of FSTL1 treatment on end diastolic pressure-volume relationship.

FIG. 8 is a graph illustrating that pulmonary congestion can be decreased following FSTL1 treatment. Heart failure (HF) group and HF group treated with FSTL1 (HF+FSTL1).

FIGS. 9A-9C collectively illustrate that FSTL1 prevents mismatch between ventricular function arterial load as a function of decreased contractility. Heart failure (HF) group and HF group treated with FSTL1 (HF+FSTL1).

FIG. 9A is a graph illustrating the effect of FSTL1 treatment on ventricular-arterial coupling ratio.

FIG. 9B is a graph illustrating the effect of FSTL1 treatment on end systolic pressure-volume relationship.

FIG. 9C is a graph illustrating the effect of FSTL1 treatment on preload recruitable stroke work.

FIGS. 10A-10B collectively illustrate that FSTL1 prevents the narrowing of pulse pressure. Heart failure (HF) group and HF group treated with FSTL1 (HF+FSTL1).

FIG. 10A is a graph illustrating the effect of FSTL1 treatment on pulmonary artery pressure.

FIG. 10B is a graph illustrating the effect of FSTL1 treatment on pulmonary pulse pressure/pulmonary systolic pressure.

FIGS. 11A-11B collectively illustrate that blood flow to the heart and skeletal muscle is increased following FSTL1 treatment.

FIG. 11A is a graph illustrating the effect of FSTL1 treatment on skeletal muscle blood flow.

FIG. 11B is a graph illustrating the effect of FSTL1 treatment on coronary blood flow.

FIGS. 12A-12C collectively illustrate the effect of FSTL1 treatment on coronary arteriole function. Impaired BK_{Ca} channel-mediated coronary arteriole dilatory capacity is attenuated by FSTL1 in remote coronary vessels. Heart failure (HF) group and HF group treated with FSTL1 (HF+FSTL1).

FIG. 12A is a graph illustrating the effect of FSTL1 treatment on % possible dilation in remote myocardium. Coronary arteriole diameter is 139.5±4.9 μm.

FIG. 12B is a graph illustrating the effect of FSTL1 treatment on % possible dilation in border zone. Coronary arteriole diameter is 139.5±4.9 μm.

FIG. 12C is a graph illustrating the effect of FSTL1 treatment on % possible dilation in infarct. Coronary arteriole diameter is 139.5±4.9 μm.

FIGS. 13A-13C collectively illustrate the effect of FSTL1 treatment on cerebral vascular function. Functional capacity is lost in second order Pial arteries in HFrEF. FSTL1 attenuates the loss of TXA2-mediated vasoconstriction. Heart failure (HF) group and HF group treated with FSTL1 (HF+FSTL1).

FIG. 13A is a graph illustrating the effect of FSTL1 treatment on % possible constriction with U46619 (thromboxane A2 agonist). Cerebral vascular diameter is 385.3±18.7 μm.

FIG. 13B is a graph illustrating the effect of FSTL1 treatment on % possible dilation with sodium nitroprusside. Cerebral vascular diameter is 385.3±18.7 μm.

FIG. 13C is a graph illustrating the effect of FSTL1 treatment on % possible dilation with NS-1619 (large-conductance calcium-activated potassium channel activator; BK_{Ca}). Cerebral vascular diameter is 385.3±18.7 μm.

FIGS. 14A-14D collectively illustrate that FSTL1 treatment improved mitochondrial function, specifically in infarct area. Mitochondrial function in subcutaneous FSTL1-treated and FSTL1-untreated myocardial-infarcted diabetic pigs was measured by high-resolution respirometry. The excised heart was analyzed in three portions, depending on distance from the infarct injury. Oxygen fluxes are shown (Y-axis) as basal or substrate-saturated respiration before adding ADP, ADP-stimulated coupled respiration with octanoyl-carnitine, glutamate, and succinate (state III), and maximal uncoupled respiration after adding the uncoupling factor FCCP.

FIG. 14A a schematic summary of an experimental design of Example 6.

FIG. 14B is a graph illustrating mitochondrial function in remote area.

FIG. 14C is a graph illustrating mitochondrial function in border zone.

FIG. 14D is a graph illustrating mitochondrial function in infarct area.

FIGS. 15A-15E collectively illustrate that FSTL1 treatment reduced necrosis in infarct area.

FIG. 15A is a graph illustrating quantification of infarct size per treatment in heart failure (HF) group and HF group treated with FSTL1 (HF+FSTL1).

FIG. 15B is a graph illustrating quantification of infarct size normalized to heart size in heart failure (HF) group and HF group treated with FSTL1 (HF+FSTL1).

FIG. 15C is a graph illustrating quantification of necrotic core volume in heart failure (HF) group and HF group treated with FSTL1 (HF+FSTL1).

FIG. 15D is a graph illustrating quantification of necrotic core volume normalized to infarct size in heart failure (HF) group and HF group treated with FSTL1 (HF+FSTL1).

FIG. 15E is a representative image of gross morphology heart section used for analysis in Example 7.

FIG. 16 is a graph illustrating FSTL1 treatment reduces lung weight. Heart failure (HF) group and HF group treated with FSTL1 (HF+FSTL1).

FIGS. 17A-17F collectively illustrate the effect of FSTL1 treatment. Heart failure (HF) group and HF group treated with FSTL1 (HF+FSTL1)

FIG. 17A is graphs illustrating that FSTL1 treatment improves systolic function.

FIG. 17B is graphs illustrating that FSTL1 treatment improves diastolic function.

FIG. 17C is a graph illustrating that FSTL1 treatment restores PA proportional pulse pressure (PAPPP).

FIG. 17D is a graph illustrating that FSTL1 treatment improves ventricular-arterial coupling.

FIG. 17E is a graph illustrating that FSTL1 treatment reduces heart rate.

FIG. 17F is a graph illustrating that FSTL1 treatment improves coronary blood flow.

### DETAILED DESCRIPTION

The present disclosure relates to, inter alia, use of human recombinant non-glycosylated or hypoglycosylated FSTL1 protein on both coronary and cerebral vascular function in cardiometabolic heart failures. Provided herein is a method of aiding heart failure treatment, the method comprising contacting the cardiac tissue with a non-glycosylated or hypoglycosylated follistatin-like 1 (FSTL1) polypeptide, wherein the heart failure can be cardiometabolic heart failure.

The following descriptions and examples illustrate embodiments of the present disclosure in detail. Although the present disclosure has been described in some details by way of illustration and example for purposes of clarity and understanding, it will be apparent that certain changes and modifications can be practiced within the scope of the appended claims.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment. It is to be understood that the present disclosure is not limited to the particular embodiments described herein and as such can vary. Those of skill in the art will recognize that there are variations and modifications of the present disclosure, which are encompassed within its scope.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the disclosure can be used in combination with any other unless specifically indicated otherwise.

### Definition

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated cases, e.g., to any commonly owned patent or application. Although any methods and materials similar or equivalent to those described herein can be used in the practice for testing of the present disclosure, the preferred materials and methods are described herein. Accordingly the terminology used herein is for the purpose of describing particular embodiments only, and is not intended to be limiting.

In this application, the use of the singular includes the plural unless specifically stated otherwise. It must be noted that, as used in the specification, the singular forms “a”, “an”, and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C”. The term “or” can be used conjunctively or disjunctively, unless the context specifically refers to a disjunctive use.

Furthermore, the use of the term “including” as well as other forms, such as “include”, “includes” and “included”, is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” can include 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

The phrase “cardiac tissue,” as used herein, refers to any tissue of the heart. Cardiac tissue includes myocardial tissue, tissue of the epicardium, and tissue of the endocardium. Cardiac tissue comprises any of the cell types found within the heart.

The phrase “epicardial-derived paracrine factor,” as used herein, can refer to any protein, polypeptide, or fragment thereof produced by the cells of the external epithelial layer of the heart capable of eliciting one or more of a physiological, protective, proliferative, and/or reparative response in the cardiac (e.g., myocardial) tissue following injury due to cardiovascular disease, myocardial infarction, or other ischemic event. In one embodiment, an epicardial-derived paracrine factor can be a component of conditioned media obtained from epicardial cell cultures.

The term “hypoglycosylated” or “non-glycosylated”, as used in the context of the instant disclosure, can refer to a protein that can be post-translationally modified with a minimal number carbohydrate moieties or which can completely lack carbohydrate moieties. In some embodiments, hypoglycosylated can refer to a protein that can completely lack any carbohydrate modification whatsoever (for example, N-linked glycans, O-linked glycans, or phospho-glycans). In another embodiment, this term can refer to a protein with decreased carbohydrate modification (such as any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%) relative to the amount of glycosylation that occurs in vivo under normal physiological conditions in mammalian cells. In another embodiment, this term can refer to a protein with decreased carbohydrate modification (such as any of about at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%) relative to the amount of glycosylation that occurs in vivo under normal physiological conditions in mammalian cells. In another embodiment, this term can refer to a protein with decreased carbohydrate modification (such as any of about at least 1-100%, 5-95%, 10-90%, 20-80%, 30-70%, 5-10%, 10-20%, 20-30%, 30-40%, 40-50%, 50-60%, 60-70%, 70-80%, 80-90%, 90-100% decreased carbohydrate modification) relative to the amount of glycosylation that occurs in vivo under normal physiological conditions in mammalian cells. In another embodiment, this term can refer to a protein with decreased carbohydrate modification (such as any of about at most 1-100%, 5-95%, 10-90%, 20-80%, 30-70%, 5-10%, 10-20%, 20-30%, 30-40%, 40-50%, 50-60%, 60-70%, 70-80%, 80-90%, 90-100% decreased carbohydrate modification) relative to the amount of glycosylation that occurs in vivo under normal physiological conditions in mammalian cells. In yet other embodiments, a hypoglycosylated protein can be engineered so that all glycosylation-competent amino acid residues (such as N-linked, O-linked, or phospho-glycan-competent amino acid residues) can be substituted with glycosylation-incompetent amino acid residues.

A “subject” or “individual” can be a vertebrate, a mammal, or a human. Mammals include, but are not limited to, farm animals, sport animals, pets, primates, rodents, mice and rats. In one aspect, a subject can be a human.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub combination was individually and explicitly disclosed herein.

### Cardiometabolic Heart Failure Overview

Heart failure is a heterogeneous disease with an age-related increase in prevalence. The increasing prevalence of heart failure in the elderly population may be attributed to the elevated number of long-term survivors after myocardial infarction who can be particularly prone to develop left ventricular dysfunction, a main driver of heart failure. In addition to age, obesity and diabetes have been identified as important risk factors for heart failure. Diabetes is one of the most relevant risk factors for heart failure, the prevalence of which is increasing worldwide. Heart failure often manifests as the first cardiovascular event in people with diabetes. Even individuals with pre-diabetes can be at a greater risk of developing heart failure. In addition, heart failure itself is emerging as an antecedent for diabetes development. Thus, diabetes and heart failure can be interrelated: diabetes increases the risk of heart failure, heart failure is highly prevalent in patients with diabetes, and heart failure may increase the risk of developing diabetes.

Three main phenotypes describe heart failure according to the measurement of the left ventricle ejection fraction. Heart failure with reduced ejection fraction (HFrEF) has ejection fraction less than or equal to about 40%. Heart failure with preserved ejection fraction (HFpEF) has ejection fraction is greater than or equal to about 50%. Heart failure with mid-range ejection fraction (HFmrEF) (other names are: HFpEF-borderline and HFpEF-improved when ejection fraction in HFrEF improves to greater than 40%) has ejection fraction about 41% to about 49% per European guidelines and about 40% to about 49% per the US guidelines. All patients with HFrEF have concomitant diastolic dysfunction; in contrast, diastolic dysfunction can occur in the absence of systolic dysfunction. Risk factors specific for HFrEF include a history of cardiovascular diseases such as myocardial infarction. The mortality rate of HFrEF patients is slightly higher than for those with HFpEF and is mainly caused by cardiovascular death.

Left ventricular dysfunction, a main driver of heart failure, can be classified into abnormalities of systolic function or abnormalities of myocardial relaxation, previously known as diastolic dysfunction. The initial step of the clinical manifestation of systolic dysfunction is an injury to myocytes, e.g., due to myocardial infarction or ischemia. Injury-induced myocyte damage and thus progressive myocyte loss provokes an inflammatory response and thereby causes ventricular remodeling. Remodeling generates an imbalanced heart wall structure with eccentric hypertrophy characterized by an increased length of myocytes. The inflammatory process triggers an excessive production of fibrotic tissues, further disturbing cardiac function by an impaired transduction of myocyte contraction into cardiac force, culminating in an uncoordinated contraction of the myocyte bundles. In many cases, HFrEF manifests as systolic dysfunction.

Diabetic cardiomyopathy is defined by a cardiac dysfunction due to a suppressed glucose metabolism and elevated fatty acid metabolism, and by the existence of an abnormal myocardial structure and performance in individuals with diabetes who do not show any symptoms/signs of coronary artery disease, valvular disease, and other cardiovascular risk factors such as hypertension and dyslipidemia.

Diabetic cardiomyopathy can be classified in four stages. During progression from stage I, with impaired myocardial relaxation but normal ejection fraction, to the final stage IV, with a clinical overt ischemia and infarct causing heart failure, muscle contraction decreases and fibrosis develops. In addition and parallel to structural changes of the heart, diabetes-associated conditions such as hyperglycemia, hyperinsulinemia, inflammation, and hyperlipidemia can alter cardiac function. In diabetic cardiomyopathy, the heart muscle shows an impaired glucose metabolism due to insulin resistance, characterized by a reduced glucose uptake, a reduced glycolytic activity, and a reduced pyruvate oxidation. Thus, glucose is limitedly available and there is an overabundance of circulating fatty acids, another principal fuel of energy next to glucose facilitating ATP production necessary for cardiac contraction, which is mainly consumed by cardiomyocytes in the case of diabetic cardiomyopathy. The resulting metabolic inflexibility and the overactive fatty acid oxidation promote a number of secondary pathways which render the heart less able to cope with increasing workloads. In particular, the fatty acid rich cardiomyocytes produce ATP less efficiently, accumulate lipids and a range of toxic intermediates, which are considered to promote pro-inflammatory and profibrotic responses, finally contributing to hypertrophy and diastolic dysfunction in diabetic cardiomyopathy. For example, ceramide can be toxic lipids, which can be synthesized when an excess of fatty acids is present. An overload of ceramide accumulates in cardiomyocytes and has profound effects on cellular signaling, such as apoptosis and insulin resistance, and facilitates ventricular modeling, fibrosis, and macrophage infiltration upon myocardial infarction.

### Compositions of the Disclosure

The present disclosure relates to, inter alia, human recombinant non-glycosylated or hypoglycosylated follistatin-like 1 (FSTL1) protein for use of both coronary and cerebral vascular function in cardiometabolic heart failures.

FSTL1, also known as follistatin-related protein 1, is an epicardial-derived paracrine factor. FSTL1 is a protein that, in humans, can be encoded by the FSTL1 gene. This gene encodes a protein with similarity to follistatin, which can be an activin-binding protein. FSTL1 contains an FS module (a follistatin-like sequence containing 10 conserved cysteine residues), a Kazal-type serine protease inhibitor domain, 2 EF hand domains, and a Von Willebrand factor type C domain (Entrez Gene: “FSTL1 follistatin-like 1).

FSTL1 can be a glycoprotein that, when non-glycosylated or hypoglycosylated, displays regenerative properties including pro-angiogenic effects and prevention of abnormal vascular remodeling. The present disclosure relates to, inter alia, use of human recombinant non-glycosylated or hypoglycosylated FSTL1 protein on both coronary and cerebral vascular function in cardiometabolic heart failures. In some embodiments, FSTL1 can attenuate the development of coronary and cerebral vascular dysfunction in cardiometabolic heart failure with reduced ejection fraction (HFrEF). In some embodiments, the cardiometabolic heart failure can be diabetic cardiomyopathy. In some embodiments, heart failure can refer to a long-term condition in which heart cannot pump blood well enough to meet the body's needs.

In an embodiment, FSTL1 can include the amino acid sequence of SEQ ID NO: 1 (NCBI Reference Sequence: NP_009016.1). Nucleic acids encoding FSTL1 are provided and contemplated within the scope of the present invention. In some embodiments, the nucleic acid can be a recombinant nucleic acid. In some embodiments, FSTL1 can be encoded by the nucleic acid of SEQ ID NO: 2 (NCBI Reference Sequence: NM_007085.4).

A polynucleic acid encoding FSTL1 can be incorporated into a vector, such as an expression vector, using standard techniques known to one of skill in the art. Methods used to ligate the DNA construct comprising a nucleic acid of interest such as FSTL1, a promoter, a terminator, and other sequences and to insert them into a suitable vector are well known in the art. Additionally, vectors can be constructed using known recombination techniques (e.g., Invitrogen Life Technologies, Gateway Technology).

In some embodiments, it can be desirable to over-express FSTL1 nucleic acids at levels far higher than currently found in naturally-occurring cells. This result can be accomplished by the selective cloning of the nucleic acids encoding those polypeptides into multicopy plasmids or placing those nucleic acids under a strong inducible or constitutive promoter. Methods for over-expressing desired polypeptides are common and well known in the art of molecular biology and examples may be found in Sambrook et al., Molecular Cloning: A Laboratory Manual, 2nd ed., Cold Spring Harbor, 2001.

A variety of host cells can be used to make a recombinant host cell that can express FSTL1. The host cell can be a cell that naturally produces FSTL1 or a cell that does not naturally produce FSTL1. For example, mammalian cells, such as, but not limited to, Chinese Hamster Ovary (CHO) cells or epicardium-derived cell cultures can be used to produce FSTL1. However, in other embodiments, cells derived from organisms that do not glycosylate proteins following translation (i.e., cells which do not post-translationally modify proteins with one or more carbohydrate moieties) can be used to produce recombinant FSTL1.

Non-limiting examples of cells that do not glycosylate proteins following translation include bacterial cells. As such, in one embodiment, the host cell can be a bacterial cell. In another embodiment, the bacterial cell can be a gram-positive bacterial cell or gram-negative bacterial cell. In another embodiment, the bacterial cell can be selected from the group consisting of E. coli, L. acidophilus, P. citrea, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., P. alcaligenes, Clostridium sp., Corynebacterium sp., and C. glutamicum cells.

FSTL1-encoding nucleic acids or vectors containing them can be inserted into a host cell (e.g., a bacterial cell) using standard techniques for expression of the encoded FSTL1 polypeptide. Introduction of a DNA construct or vector into a host cell can be performed using techniques such as transformation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. General transformation techniques are well known in the art (see, e.g., Current Protocols in Molecular Biology (F. M. Ausubel et al. (eds) Chapter 9, 1987; Sambrook et al., Molecular Cloning: A Laboratory Manual, 3rd ed., Cold Spring Harbor, 2001; and Campbell et al., Curr Genet, 16:53-56, 1989, which are each hereby incorporated by reference in their entireties, particularly with respect to transformation methods). The introduced nucleic acids can be integrated into chromosomal DNA of the host cell or maintained as extrachromosomal replicating sequences. In yet another embodiment, an FSTL1 polypeptide can be produced in a host cell via delivery of chemically modified mRNAs encoding the mutated FSTL1 glycosylation-deficient polypeptide. (see Modified mRNA directs the fate of heart progenitor cells and induces vascular regeneration after myocardial infarction. Zangi L, et al. Nat Biotechnol. 2013 October; 31 (10): 898-907, incorporated herein by reference in its entirety). Chemically modified RNAs, also referred to herein as modRNAs, can include, for example, modifications of phosphate into phosphorothioate internucleotidic linkages, modifications of the 2′-hydroxyl group of ribose, or other modifications to the phosphate backbone or sugar moieties of mRNA.

In some embodiments, FSTL1 can be a non-glycosylated or hypoglycosylated FSTL1. Non-glycosylated FSTL1 or hypoglycosylated can be obtained by producing recombinant FSTL1 in host cells that naturally do not post-translationally modify proteins with carbohydrate moieties (such as bacteria, e.g. E. coli) or which have been engineered such that they can be unable to post-translationally modify proteins with carbohydrate moieties. Alternatively, non-glycosylated or hypoglycosylated FSTL1 can be produced in mammalian or other eukaryotic cells that normally post-translationally modify proteins with carbohydrate moieties but which have been treated with one or more glycosylation inhibitors. Suitable glycosylation inhibitors include, without limitation, tunicamycin (which blocks all N-glycosylation of proteins), streptovirudin, mycospocidin, amphomycin, tsushimycin, antibiotic 24010, antibiotic MM 19290, bacitracin, corynetoxin, showdomycin, duimycin, 1-deoxymannonojirimycin, deoxynojirimycin, N-methyl-1-dexoymannojirimycin, brefeldin A, a glucose analog, a mannose analog, 2-deoxy-D-glucose, 2-deoxyglucose, D-(+)-mannose, D-(+) galactose, 2-deoxy-2-fluoro-D-glucose, 1,4-dideoxy-1,4-imino-D-mannitol (DIM), fluoroglucose, fluoromannose, UDP-2-deoxyglucose, GDP-2-deoxyglucose, a hydroxymethylglutaryl-CoA reductase inhibitor, 25-hydroxycholesterol, hydroxycholesterol, swainsonine, cycloheximide, puromycin, actinomycin D, monensin, m-Chlorocarbonyl-cyanide phenylhydrazone (CCCP), compactin, dolichyl-phosphoryl-2-deoxyglucose, N-Acetyl-D-Glucosamine, hygoxanthine, thymidine, cholesterol, glucosamine, mannosamine, castanospermine, glutamine, bromoconduritol, conduritol epoxide, a conduritol derivative, aglycosylmethyl-p-nitrophenyltriazene, β-Hydroxynorvaline, threo-β-fluoroasparagine, D-(+)-Gluconic acid δ-lactone, di(2-ethyl hexyl)phosphate, tributyl phosphate, dodecyl phosphate, 2-dimethylamino ethyl ester of (diphenyl methyl)-phosphoric acid, [2-(diphenyl phosphinyloxy)ethyl]trimethyl ammonium iodide, iodoacetate, 2-deoxy-D-glucose, and fluoroacetate.

Alternatively, in other embodiments, recombinant FSTL1 can be engineered such that it can be unable to be glycosylated when produced using a eukaryotic or other glycosylation-competent host cell. In most biological contexts, glycosylation can be either N-linked or O-linked. The N-linked glycosylation process occurs in eukaryotes and widely in archaea, but very rarely in eubacteria. In N-linked glycosylation, glycans (i.e. carbohydrate-containing moieties) can be attached to the nitrogen atom of an asparagine or arginine amino acid side-chain. N-linked glycans can be almost always attached to the nitrogen atom of an asparagine (Asn) side chain that can be present as a part of Asn-X-Ser/Thr consensus sequence, where X can be any amino acid except proline (Pro), serine (Ser), and threonine (Thr). O-linked glycosylation can be a form of glycosylation that occurs in the Golgi apparatus in eukaryotes. In O-linked glycosylation, glycans can be attached to the hydroxyl oxygen of serine, threonine, tyrosine, hydroxylysine, or hydroxyproline amino acid side-chains.

Consequently, in some embodiments, recombinant FSTL1 can be engineered so that it can be unable to be N-linked glycosylated. In this instance, some or all glycosylation-competent arginine or asparagine amino acids in the polypeptide sequence can be substituted with a glycosylation-incompetent amino acid (for example, glutamine). In other embodiments, recombinant FSTL1 can be engineered so that it can be unable to be O-linked glycosylated. In this instance, all glycosylation-competent serine, threonine, tyrosine, hydroxylysine, or hydroxyproline residues in the polypeptide sequence can be substituted with a glycosylation-incompetent amino acid (for example, alanine). In yet further embodiments, recombinant FSTL1 can be engineered so that it can be unable to be either O-linked glycosylated or N-linked glycosylated by substituting all glycosylation-competent amino acids with glycosylation-incompetent amino acids. In a further embodiment, one or more asparagine (N) residues located at positions X144, X180, X175, and/or X223 in the FSTL1 amino acid sequence can be substituted with a glycosylation-incompetent amino acid (such as, but not limited to, glutamine (Q)). Engineered glycosylation-incompetent FSTL1 can be produced in host cells via transfection of a plasmid, viral vector carrying a gene encoding a glycosylation-incompetent FSTL1 or chemically synthetized mRNA or mRNA-mimetics. Alternatively, a gene encoding a glycosylation-incompetent FSTL1 can be integrated into a chromosome of the host cell under the control of an inducible or constitutively-expressing promoter. In yet another embodiment, a glycosylation incompetent FSTL1 polypeptide can be produced in a host cell via delivery of modified mRNAs encoding a glycosylation incompetent FSTL1 polypeptide.

### Methods of the Disclosure

The present disclosure relates to, inter alia, use of human recombinant non-glycosylated or hypoglycosylated FSTL1 protein on both coronary and cerebral vascular function in cardiometabolic heart failures. The present disclosure also relates to methods of aiding heart failure treatment, the methods including contacting the cardiac tissue with a non-glycosylated or hypoglycosylated follistatin-like 1 (FSTL1) polypeptide, wherein the heart failure can be cardiometabolic heart failure. The present disclosure also relates to methods of treating cardiometabolic heart failure, the methods including contacting cardiac tissue with a non-glycosylated follistatin-like 1 (FSTL1) polypeptide. The present disclosure further relates to methods of repairing cardiac tissue following cardiometabolic heart failure, the methods including contacting the cardiac tissue with a non-glycosylated follistatin-like 1 (FSTL1) polypeptide.

In any of the methods described herein, the cardiometabolic heart failure can be a heart failure with reduced ejection fraction (HFrEF). In some embodiments, the cardiometabolic heart failure can be a diabetic cardiomyopathy. In some embodiments, the FSTL1 can decrease heart failure mortality. In some embodiments, the FSTL1 can attenuate the development of coronary and cerebral vascular dysfunction. In some embodiments, the FSTL1 can improve cardiac function. In some embodiments, the FSTL1 can reduce heart rate. In some embodiments, the FSTL1 does not alter left ventricular volume and systolic or diastolic volume. In some embodiments, the FSTL1 can prevent left ventricular diastolic dysfunction. In some embodiments, the FSTL1 can improve ventricular-vascular interactions. In some embodiments, the FSTL1 can decrease pulmonary congestion. In some embodiments, the FSTL1 can prevent mismatch between ventricular function and arterial load as a function of decreased contractility. In some embodiments, the FSTL1 can prevent the narrowing of pulse pressure. In some embodiments, the FSTL1 can improve coronary and/or peripheral vascular function. In some embodiments, the FSTL1 can increase blood flow to the heart and skeletal muscles. In some embodiments, the FSTL1 can attenuate impaired large-conductance voltage- and Ca₂⁺-activated K⁺ channel (BKCa) channel-mediated coronary arteriole dilatory capacity in remote coronary vessels. BKCa can be an important regulator of membrane excitability in a wide variety of cells and tissues. In some embodiments, the FSTL1 can attenuate the loss of thromboxane A2 (TXA2)-mediated vasoconstriction. TXA2 can be a known vasoconstrictor and get activated during times of tissue injury and inflammation. In some embodiments, the FSTL1 can improve mitochondrial function. In some embodiments, the FSTL1 can reduce necrosis in infarct area. In some embodiments, the FSTL1 can reduce lung weight.

The injury to the cardiac (e.g., myocardial) tissue can be associated with any number of diseases or conditions known to affect the heart or circulatory system and include, without limitation, coronary heart disease, cardiomyopathy, ischemic heart disease, heart failure, inflammatory heart disease, valvular heart disease and aneurysm. In one embodiment, the injury can be caused by myocardial infarction (MI; such as acute myocardial infarction (AMI)). In another embodiment, the injury can be caused by an ischemic event followed by reperfusion.

Repair of injured cardiac (e.g., myocardial) tissue can comprise increasing the number of cardiomyocytes that can be indirectly measure in the live subject by several methods of imaging (like delayed enhance MRI, DE-MRI) as decreased in myocardial infarct size. See for example: (Hendel R C et al, JACC 48 (7); 1475-97) and (Sardella G et al, JACC 2009; 53 (4): 309-15, incorporated herein by reference in its entirety). In some embodiments, contacting the cardiac (e.g., myocardial) tissue with an epicardial-derived paracrine factor (such as non-glycosylated FSTL1) can result in any of about a 2%, 5%, 10%, 15%, 20%, 30%, 40% 50%, 60%, 90%, 100%, or about a 5-10%, 10-20%, 20-30%, 30-40%, 40-50%, 50-60%, 60-70%, 70-80%, 80-90%, 90-100% recovery of lost muscle and reduction of infarct size. In other embodiments, contacting the cardiac (e.g., myocardial) tissue with an epicardial-derived paracrine factor (such as non-glycosylated FSTL1) can result in any of about at least 2%, 5%, 10%, 15%, 20%, 30%, 40% 50%, 60%, 90%, 100%, or about a 5-10%, 10-20%, 20-30%, 30-40%, 40-50%, 50-60%, 60-70%, 70-80%, 80-90%, 90-100% recovery of lost muscle and reduction of infarct size. In other embodiments, contacting the cardiac (e.g., myocardial) tissue with an epicardial-derived paracrine factor (such as non-glycosylated FSTL1) can result in any of about at most 2%, 5%, 10%, 15%, 20%, 30%, 40% 50%, 60%, 90%, 100%, or about a 5-10%, 10-20%, 20-30%, 30-40%, 40-50%, 50-60%, 60-70%, 70-80%, 80-90%, 90-100% recovery of lost muscle and reduction of infarct size.

An epicardial-derived paracrine factor (such as non-glycosylated FSTL1) can be delivered using various methods known in the art. In some embodiments, the FSTL1 can be delivered by systemically. In some embodiments, the FSTL1 can be delivered endocardially. In some embodiments, the endocardial delivery can be via a catheter. In some embodiments, the FSTL1 can be delivered epicardially. In some embodiments, the cardiac tissue can be contacted from one or more of an epicardial site, an endocardial site, and/or through direct injection into the myocardium. In some embodiments, the FSTL1 can be delivered using a drug-eluting stent. In some embodiments, the FSTL1 can be delivered by a hydrogel embedded or seeded with the FSTL1. In some embodiments, the FSTL1 can be delivered by a collagen patch embedded or seeded with the FSTL1. In some embodiments, the FSTL1 can be delivered via an osmotic pump. In some embodiments, the FSTL1 can be delivered as a single or several subcutaneous bolus. In some embodiments, the FSTL1 can be delivered by a coronary infusion. In some embodiments, the FSTL1 can be expressed in the heart by use of modified RNAs (modRNAs). In some embodiments, the FSTL1 can be expressed by genomic editing.

In some embodiments of any of the methods disclosed herein, the epicardial-derived paracrine factor (such as non-glycosylated FSTL1) can be infused, seeded, or embedded into a 3D collagen based-patch. The collagen based patch can then be contacted directly to the epicardium or an injured area of myocardium (such as an area of the myocardium exposed to an ischemic event, such as myocardial infarction). The 3D collagen can be applied to the epicardium or myocardium via suturing or by any other means known in the art for contacting the patch to the injured tissue.

In yet other embodiments, the epicardial-derived paracrine factor (such as non-glycosylated FSTL1) can be a component of a hydrogel that can be delivered to the epicardium, to the endocardium, or to an injured area of myocardium (by, for example, catheter technology; Koudstaal et al., J. of Cardiovasc. Trans. Res. (2014) 7:232-241, incorporated herein by reference in its entirety).

In some embodiments, the effect on the cardiac (e.g., myocardial) tissue can be achieved with placement of non-glycosylated or hypoglycosylated FSTL1 endocardially into the heart by percutaneous catheter delivery systems, for example as the systems available developed by BioCardia (www.biocardia.com).

In some embodiments, the effect on the cardiac (e.g., myocardial) tissue can be achieved with placement of non-glycosylated or hypoglycosylated FSTL1 epicardially into the heart using catheter devices similar to those used in other applications (for example Epicardial Catheter System™, St. Jude Medical).

In some embodiments, the effect on the cardiac (e.g., myocardial) tissue can be achieved with placement of non-glycosylated or hypoglycosylated FSTL1 when impregnated in drug-diluting stents (for example, those available from Abbott Laboratories or Biosensors International, among others).

In some embodiments, the effect on the cardiac (e.g., myocardial) tissue can be achieved with placement of non-glycosylated or hypoglycosylated FSTL1 systemically, using approved formulation.

In some embodiments, the effect on the cardiac (e.g., myocardial) tissue can be achieved with placement of hypoglycosylated FSTL1 can be achieved by the use of compound or drugs that inhibit the glycosylation of the endogenous glycosylated FSTL1 protein, which can be readily available and known to one of skill in the art. Accordingly, any of the methods described herein can further comprise an inhibitor of FSTL1 glycosylation. In some embodiments, the inhibitor of FSTL1 glycosylation can be selected from the group consisting of tunicamycin, streptovirudin, mycospocidin, amphomycin, tsushimycin, antibiotic 24010, antibiotic MM 19290, bacitracin, corynetoxin, showdomycin, duimycin, 1-deoxymannonojirimycin, deoxynojirimycin, N-methyl-1-dexoymannojirimycin, brefeldin A, a glucose analog, a mannose analog, 2-deoxy-D-glucose, 2-deoxyglucose, D-(+)-mannose, D-(+) galactose, 2-deoxy-2-fluoro-D-glucose, 1,4-dideoxy-1,4-imino-D-mannitol (DIM), fluoroglucose, fluoromannose, UDP-2-deoxyglucose, GDP-2-deoxyglucose, a hydroxymethylglutaryl-CoA reductase inhibitor, 25-hydroxycholesterol, hydroxycholesterol, swainsonine, cycloheximide, puromycin, actinomycin D, monensin, m-Chlorocarbonyl-cyanide phenylhydrazone (CCCP), compactin, dolichyl-phosphoryl-2-deoxyglucose, N-Acetyl-D-Glucosamine, hygoxanthine, thymidine, cholesterol, glucosamine, mannosamine, castanospermine, glutamine, bromoconduritol, conduritol epoxide, a conduritol derivative, aglycosylmethyl-p-nitrophenyltriazene, β-Hydroxynorvaline, threo-β-fluoroasparagine, D-(+)-Gluconic acid δ-lactone, di(2-ethyl hexyl)phosphate, tributyl phosphate, dodecyl phosphate, 2-dimethylamino ethyl ester of (diphenyl methyl)-phosphoric acid, [2-(diphenyl phosphinyloxy)ethyl]trimethyl ammonium iodide, iodoacetate, 2-deoxy-D-glucose, and fluoroacetate. In some embodiments, the inhibitor of FSTL1 glycosylation can be tunicamycin.

In some embodiments, the effect on the cardiac (e.g., myocardial) tissue can be achieved with placement of non-glycosylated or hypoglycosylated FSTL1 can be achieved by introduction of modRNAs encoding for specific mutagenesis targeting N-glycosylation sites in the FSTL1 mRNA sequence.

In some embodiments, the effect on the cardiac (e.g., myocardial) tissue can be achieved with placement of non-glycosylated or hypoglycosylated FSTL1 can be achieved by genome editing using CRISPR/Cas9 technology or similar, (see for example Genome editing with Cas9 in adult mice corrects a disease mutation and phenotype. Hao Yin, et al. Nature Biotechnology 32, 551-553 (2014) doi: 10.1038/nbt.2884, incorporated by reference herein in its entirety).

In some other embodiments, the effect on the cardiac (e.g., myocardial) tissue can be achieved with delivery of small molecule mimetic of non-glycosylated or hypoglycosylated FSTL1.

The injured cardiac (e.g., myocardial) tissue can be contacted with any of the epicardial-derived paracrine factor (such as non-glycosylated FSTL1) compositions (such as pharmaceutical compositions) disclosed herein before, during, or subsequent to the injury to the cardiac (e.g., myocardial) tissue. In some embodiments, the cardiac (e.g., myocardial) tissue can be contacted with the epicardial-derived paracrine factor composition in a subject deemed at risk for cardiometabolic disease, cardiovascular disease, myocardial infarction, or another myocardial ischemic event in order to mitigate or prevent injury to the myocardium by the event. In other embodiments, the cardiac (e.g., myocardial) tissue can be contacted with the epicardial-derived paracrine factor composition immediately following the onset of an ischemic event caused by cardiovascular disease or myocardial infarction, such as about 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 45 minutes, 1 hour, 1.5 hours, 2 hours, 2.5 hours, 3 hours, 3.5 hours, 4 hours, 4.5 hours, 5 hours 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, or 12 hours or more (inclusive of all time periods falling in between these values). In some embodiments, the composition can be administered less than 1 minute after the cardiac injury. Alternatively, in other embodiments, the cardiac (e.g., myocardial) tissue can be contacted with the epicardial-derived paracrine factor composition subsequent to the injury, such as at least 12 hours, 13 hours, 14 hours, 15 hours, 16 hours, 17 hours, 18 hours, 19 hours, 20 hours, 21 hours, 22 hours, 23 hours, 24 hours or 1 day, 2 days, 3 days, 4 days, 5 days, 6 days, 7 days, 8 days, 9 days, 10 days, 11 days, 12 days, 13 days, 14 days, three weeks, one month, 2 months, 3 months, 4 months, 6 months, 7 months, 8 months, 9 months, 10 months, 11 months, or one or more years (inclusive of all time periods falling in between these values) following the onset of an ischemic event caused by cardiovascular disease or myocardial infarction.

Any of the methods of treating injuries to cardiac (e.g., myocardial) tissue disclosed herein can result in increased survival in a subject following injury. As used herein, increased survival includes, e.g., at least about a 5% (e.g., at least about 10%, 15%, 20%, 25%, 30%, 40%, 45%, 50%, 60%, 70%, 80%, 90%, 100%, 110%, 120%, 130%, 140%, 150% or more than 200% or greater) increase in the survival of a subject compared to relative survival in subjects who have not been subject to the instantly described methods. Survival time can be measured, e.g., in days, weeks, months, or years. In some embodiments, contacting injured cardiac (e.g., myocardial) tissue with an epicardial-derived paracrine factor in accordance with any of the methods described herein can prolong the survival of subject by at least six months, seven months, eight months, nine months, 10 months, 12 months, 18 months, 24 months, 36 months, or more.

In some embodiments, repair of injured cardiac (e.g., myocardial) tissue can comprise decreased or attenuated fibrosis in cardiac (e.g., myocardial) tissue compared to the amount of fibrosis in cardiac (e.g., myocardial) tissue that can be not contacted by an epicardial-derived paracrine factor following an injury. In some embodiments, contacting the cardiac (e.g., myocardial) tissue with an epicardial-derived paracrine factor (such as non-glycosylated FSTL1) can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or greater reduction in fibrosis in cardiac (e.g., myocardial) tissue, inclusive of all values falling in between these percentages. In some embodiments, contacting the cardiac (e.g., myocardial) tissue with an epicardial-derived paracrine factor (such as non-glycosylated FSTL1) can result in any of about at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% reduction in fibrosis in cardiac (e.g., myocardial) tissue, inclusive of all values falling in between these percentages. In some embodiments, contacting the cardiac (e.g., myocardial) tissue with an epicardial-derived paracrine factor (such as non-glycosylated FSTL1) can result in any of about at least 1-100%, 5-95%, 10-90%, 20-80%, 30-70%, 5-10%, 10-20%, 20-30%, 30-40%, 40-50%, 50-60%, 60-70%, 70-80%, 80-90%, or 90-100% reduction in fibrosis in cardiac (e.g., myocardial) tissue, inclusive of all values falling in between these percentages. Assessment of cardiomyocyte fibrosis can be routine in the art and can be measured by DE-MRI, or by histologic examination of cardiac (e.g., myocardial) tissue (post-mortem, or biopsy). In some embodiments, contacting the cardiac (e.g., myocardial) tissue with an epicardial-derived paracrine factor (such as non-glycosylated FSTL1) can result in any of about at most 1-100%, 5-95%, 10-90%, 20-80%, 30-70%, 5-10%, 10-20%, 20-30%, 30-40%, 40-50%, 50-60%, 60-70%, 70-80%, 80-90%, or 90-100% reduction in fibrosis in cardiac (e.g., myocardial) tissue, inclusive of all values falling in between these percentages. Assessment of cardiomyocyte fibrosis can be routine in the art and can be measured by DE-MRI, or by histologic examination of cardiac (e.g., myocardial) tissue (post-mortem, or biopsy).

In some embodiments, contacting the cardiac (e.g., myocardial) tissue with an epicardial-derived paracrine factor (such as non-glycosylated FSTL1) can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or greater improvement of cardiac function, inclusive of all values falling in between these percentages. In some embodiments, contacting the cardiac (e.g., myocardial) tissue with an epicardial-derived paracrine factor (such as non-glycosylated FSTL1) can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, or greater reduction of heart rate when compared with a subject not treated with an epicardial-derived paracrine factor (such as non-glycosylated FSTL1), inclusive of all values falling in between these percentages. In some embodiments, contacting the cardiac (e.g., myocardial) tissue with an epicardial-derived paracrine factor (such as non-glycosylated FSTL1) does not alter left ventricular end systolic or diastolic volume.

In some embodiments, contacting the cardiac (e.g., myocardial) tissue with an epicardial-derived paracrine factor (such as non-glycosylated FSTL1) prevents left ventricular diastolic dysfunction. In some embodiments, contacting the cardiac (e.g., myocardial) tissue with an epicardial-derived paracrine factor (such as non-glycosylated FSTL1) can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, or greater improvement of left ventricular end systolic pressure, inclusive of all values falling in between these percentages. In some embodiments, contacting the cardiac (e.g., myocardial) tissue with an epicardial-derived paracrine factor (such as non-glycosylated FSTL1) can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, or greater reduction of left ventricular end diastolic pressure when compared with a subject not treated with an epicardial-derived paracrine factor (such as non-glycosylated FSTL1), inclusive of all values falling in between these percentages. In some embodiments, contacting the cardiac (e.g., myocardial) tissue with an epicardial-derived paracrine factor (such as non-glycosylated FSTL1) can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, or greater reduction of end diastolic pressure-volume relationship when compared with a subject not treated with an epicardial-derived paracrine factor (such as non-glycosylated FSTL1), inclusive of all values falling in between these percentages.

In some embodiments, contacting the cardiac (e.g., myocardial) tissue with an epicardial-derived paracrine factor (such as non-glycosylated FSTL1) improves ventricular-vascular interactions. In some embodiments, contacting the cardiac (e.g., myocardial) tissue with an epicardial-derived paracrine factor (such as non-glycosylated FSTL1) can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, or greater reduction of pulmonary congestion when compared with a subject not treated with an epicardial-derived paracrine factor (such as non-glycosylated FSTL1), inclusive of all values falling in between these percentages. In some embodiments, contacting the cardiac (e.g., myocardial) tissue with an epicardial-derived paracrine factor (such as non-glycosylated FSTL1) can prevent mismatch between ventricular function and arterial load as a function of decreased contractility. In some embodiments, contacting the cardiac (e.g., myocardial) tissue with an epicardial-derived paracrine factor (such as non-glycosylated FSTL1) can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, or greater reduction of ventricular-arterial coupling ratio when compared with a subject not treated with an epicardial-derived paracrine factor (such as non-glycosylated FSTL1), inclusive of all values falling in between these percentages. In some embodiments, contacting the cardiac (e.g., myocardial) tissue with an epicardial-derived paracrine factor (such as non-glycosylated FSTL1) can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, or greater improvement of end systolic pressure-volume relationship, inclusive of all values falling in between these percentages. In some embodiments, contacting the cardiac (e.g., myocardial) tissue with an epicardial-derived paracrine factor (such as non-glycosylated FSTL1) can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, or greater improvement of preload recruitable stroke work, inclusive of all values falling in between these percentages. In some embodiments, contacting the cardiac (e.g., myocardial) tissue with an epicardial-derived paracrine factor (such as non-glycosylated FSTL1) prevents narrowing of pulse pressure.

In some embodiments, contacting the cardiac (e.g., myocardial) tissue with an epicardial-derived paracrine factor (such as non-glycosylated FSTL1) can increase blood flow to the heart and skeletal muscle by about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, or greater, inclusive of all values falling in between these percentages. In some embodiments, contacting the cardiac (e.g., myocardial) tissue with an epicardial-derived paracrine factor (such as non-glycosylated FSTL1) can attenuate impaired BK_{Ca} channel-mediated coronary arteriole dilatory capacity in remote coronary vessels. In some embodiments, contacting the cardiac (e.g., myocardial) tissue with an epicardial-derived paracrine factor (such as non-glycosylated FSTL1) can attenuate the loss of TXA₂-mediated vasoconstriction.

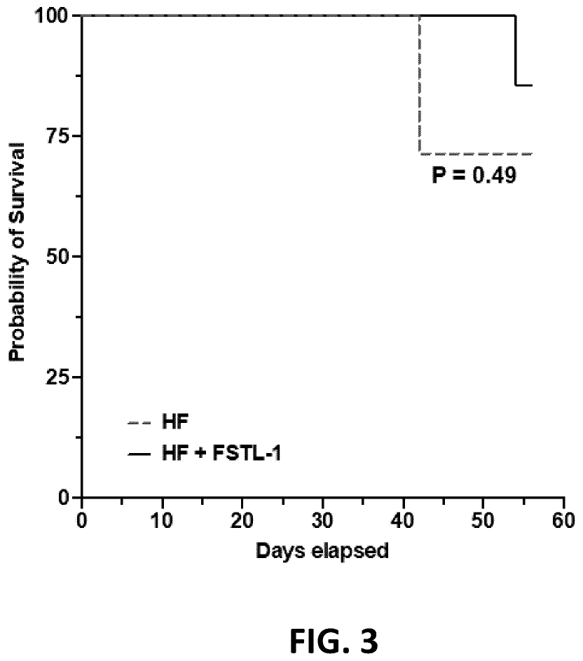

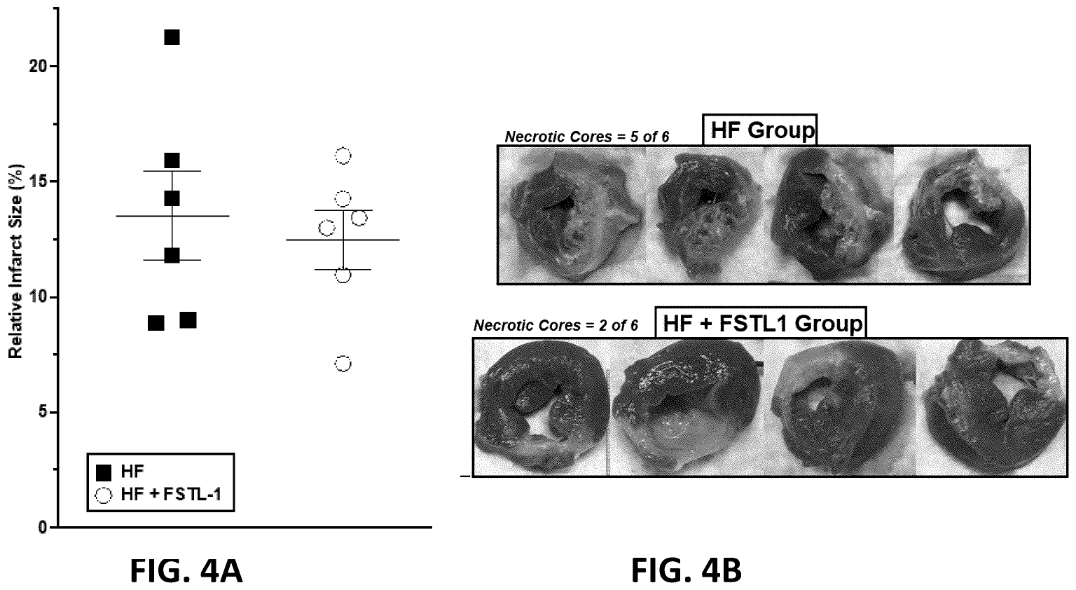

In an embodiment, the treatment with the FSTL1 of the present disclosure can prevent coronary and cerebral vascular dysfunction (Example 2). For example, the treatment with non-glycosylated FSTL1 can increase probability of survival and can reduce infarct size in Ossabaw swine with cardiometabolic heart failure with reduced ejection fraction. FIGS. 4A-4B illustrate the effect of treatment with non-glycosylated FSTL1 on size of infarct. FIG. 3 illustrates the effect of treatment with non-glycosylated FSTL1 on survival probability.

In an embodiment, the treatment with the FSTL1 of the present disclosure can increase probability of survival and can reduce infarct size (Example 3). For example, the treatment with non-glycosylated FSTL1 can increase probability of survival and can reduce infarct size in Ossabaw swine with cardiometabolic heart failure with reduced ejection fraction. FIGS. 4A-4B illustrate the effect of treatment with non-glycosylated FSTL1 on size of infarct. FIG. 3 illustrates the effect of treatment with non-glycosylated FSTL1 on survival probability.

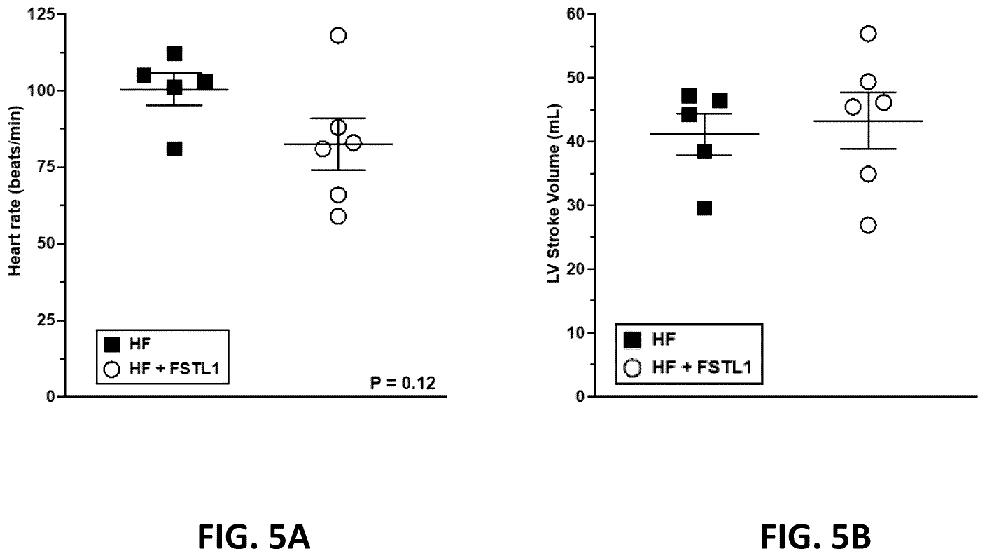

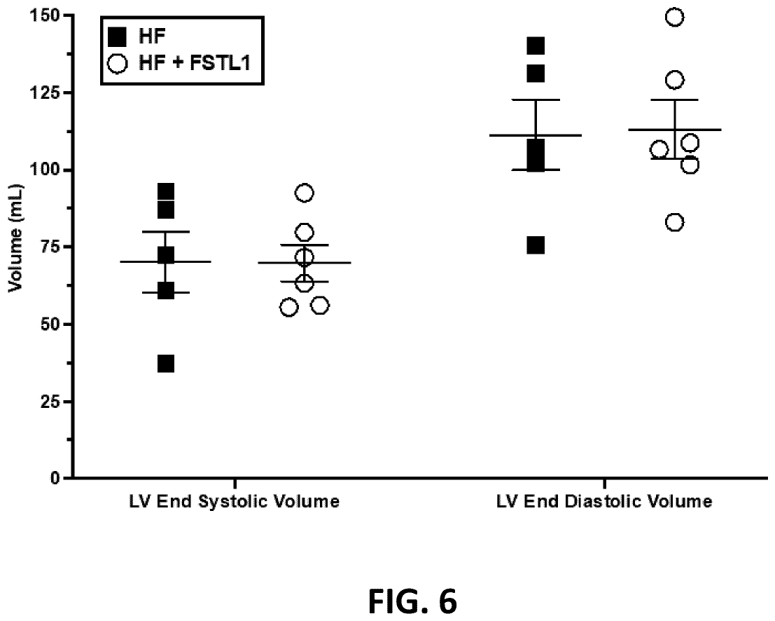

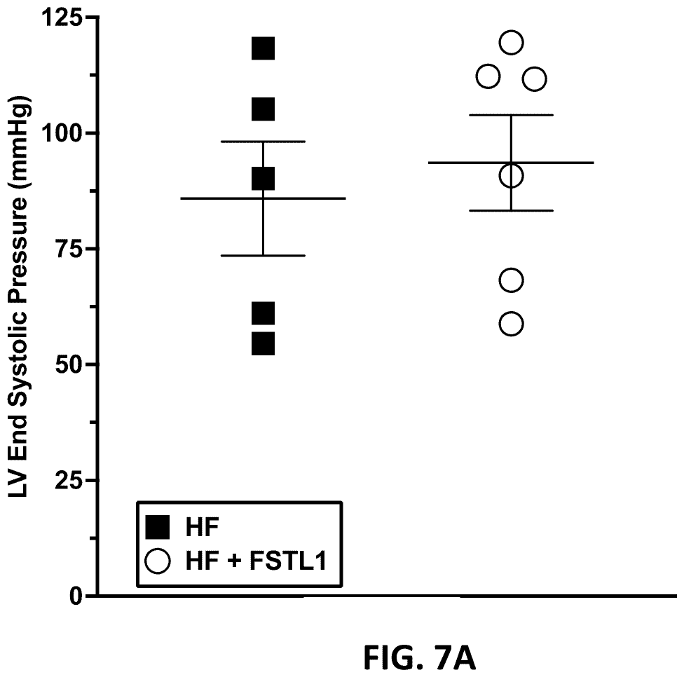

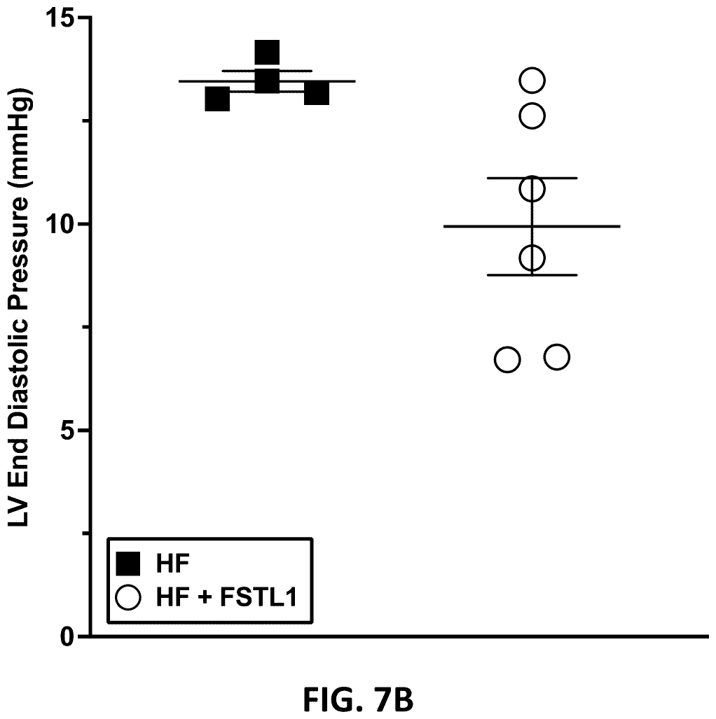

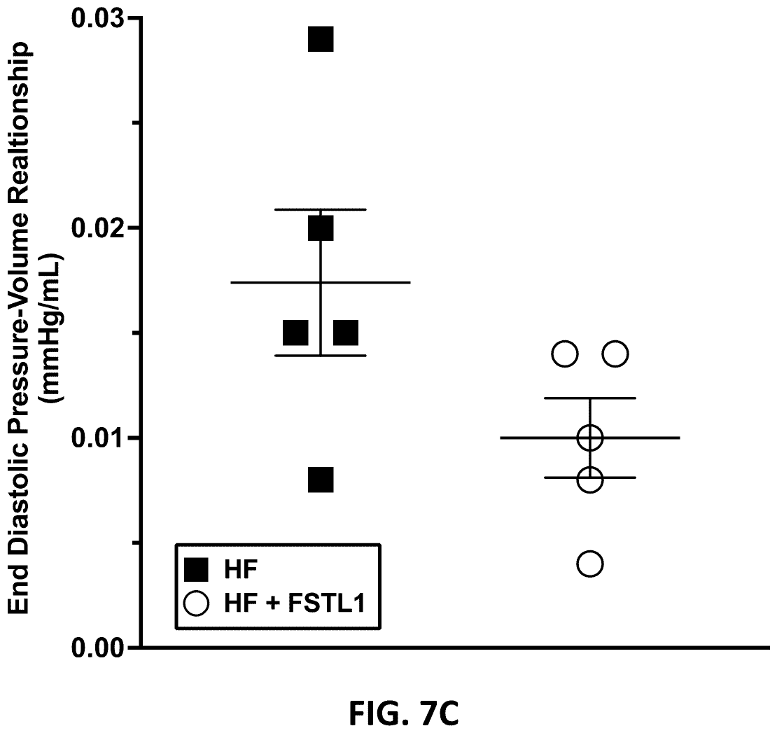

In an embodiment, the FSTL1 of the present disclosure can improve cardiac function (Example 4). For example, the treatment with non-glycosylated FSTL1 can improve cardiac function in Ossabaw swine with cardiometabolic heart failure with reduced ejection fraction. FIGS. 5A-5B illustrate that heart rate can be reduced following FSTL1 treatment. FIG. 6 illustrates that FSTL1 treatment does not alter left ventricular end systolic or diastolic volume. FIGS. 7A-7C illustrate that FSTL1 can prevent left ventricular diastolic dysfunction.

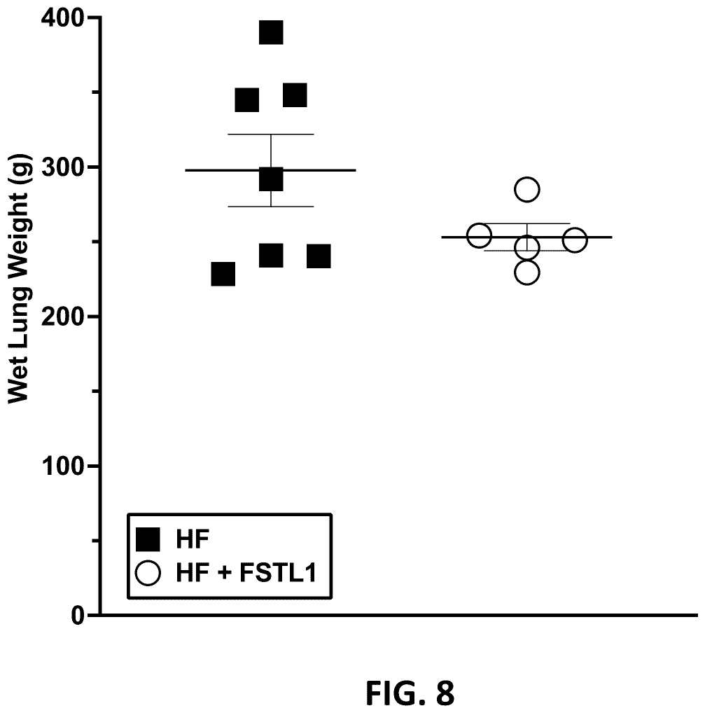

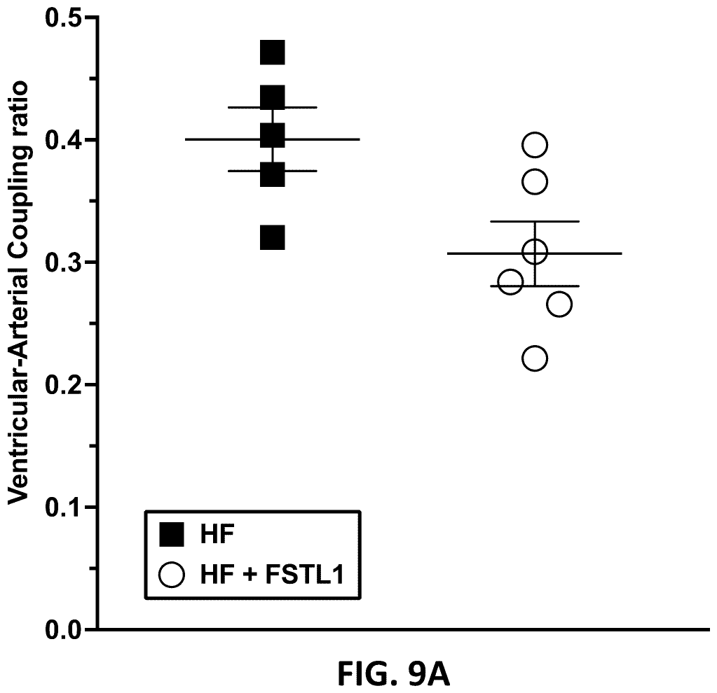

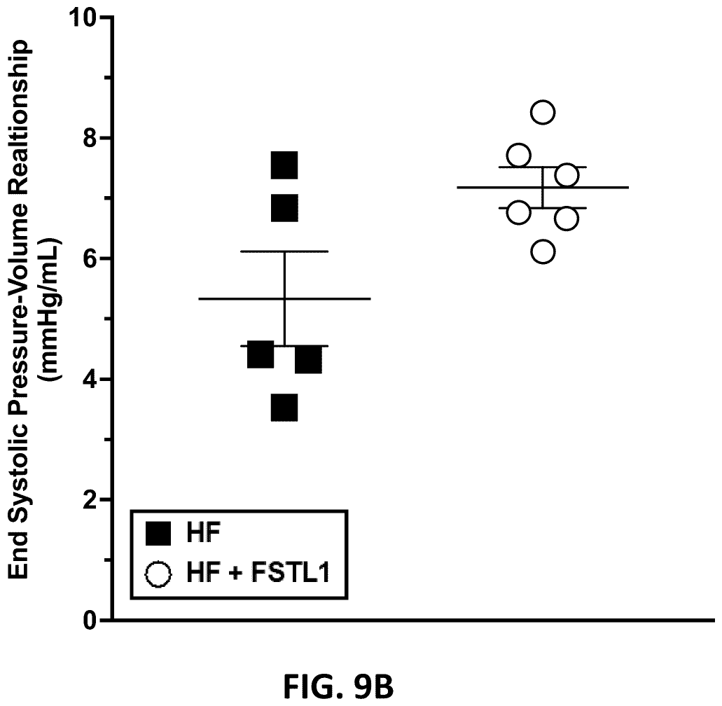

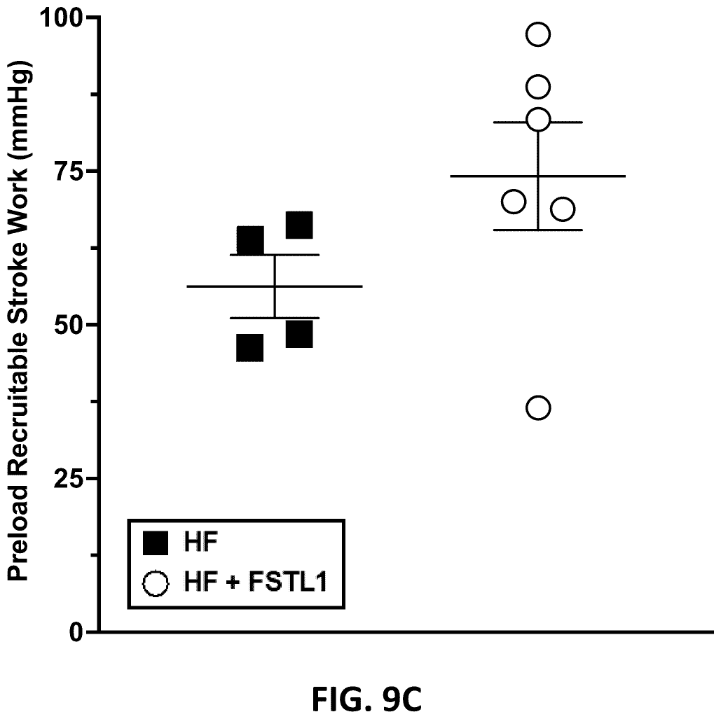

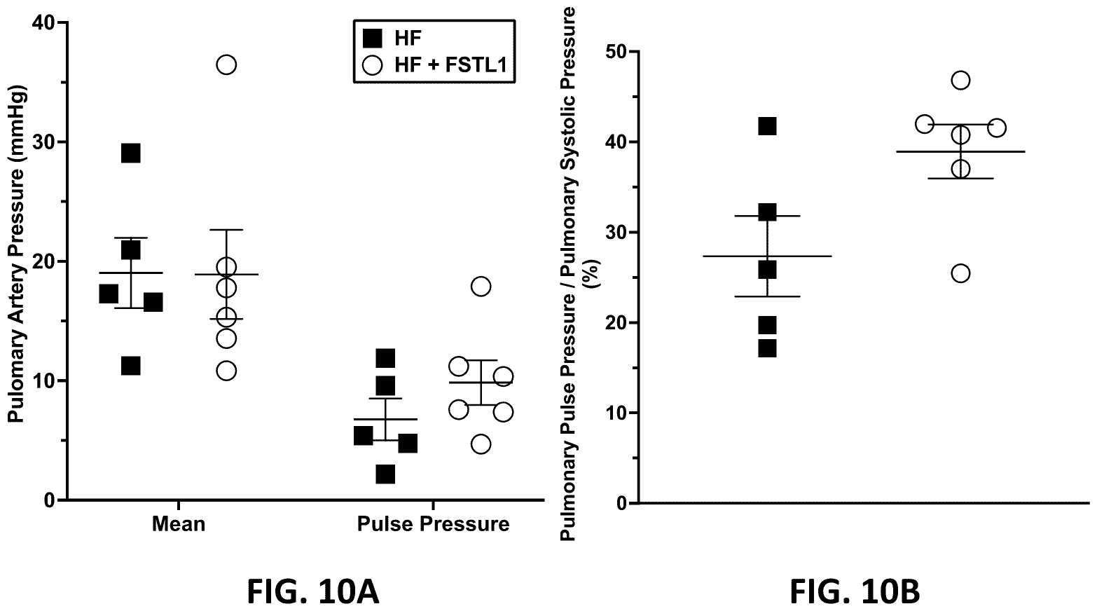

In an embodiment, the treatment with the FSTL1 of the present disclosure can improve ventricular-vascular interactions (Example 5). For example, the treatment with non-glycosylated FSTL1 can improve ventricular-vascular interactions in Ossabaw swine with cardiometabolic heart failure with reduced ejection fraction. FIG. 8 illustrates that pulmonary congestion can be decreased following FSTL1 treatment. FIGS. 9A-9C collectively illustrate that FSTL1 can prevent mismatch between ventricular function arterial load as a function of decreased contractility. FIG. 9A illustrates the effect of FSTL1 treatment on ventricular-arterial coupling ratio. FIG. 9B illustrates the effect of FSTL1 treatment on end systolic pressure-volume relationship. FIG. 9C illustrates the effect of FSTL1 treatment on preload recruitable stroke work. FIGS. 10A-10B illustrate that FSTL1 can prevent the narrowing of pulse pressure. FIG. 10A illustrates the effect of FSTL1 treatment on pulmonary artery pressure. FIG. 10B illustrates the effect of FSTL1 treatment on pulmonary pulse pressure/pulmonary systolic pressure.

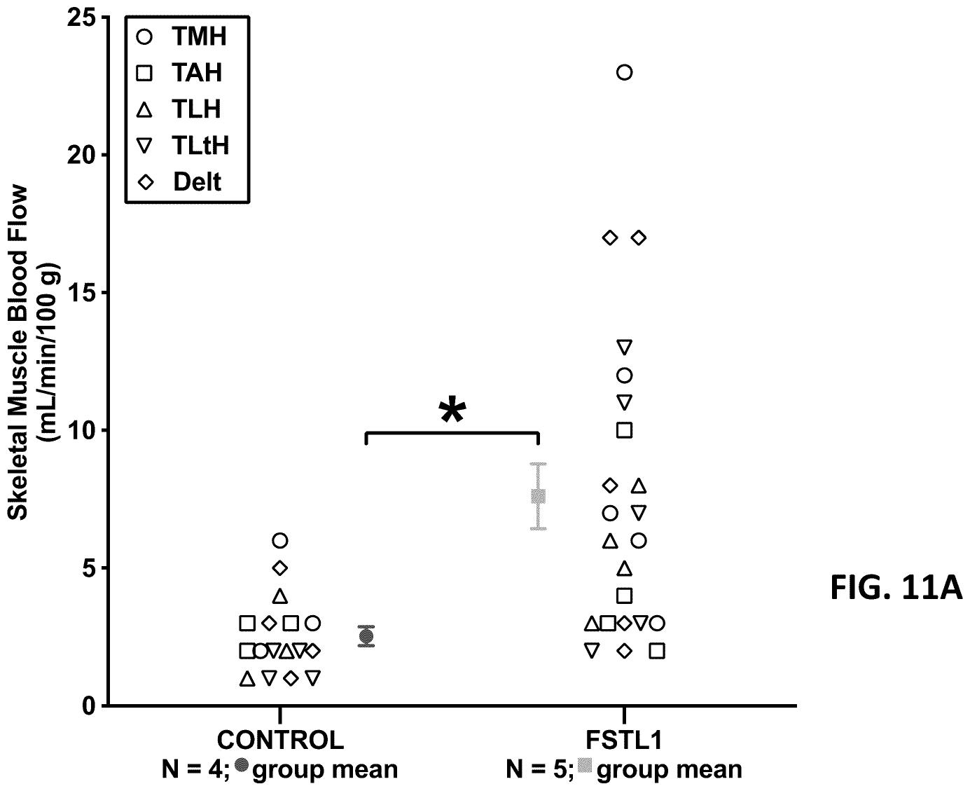

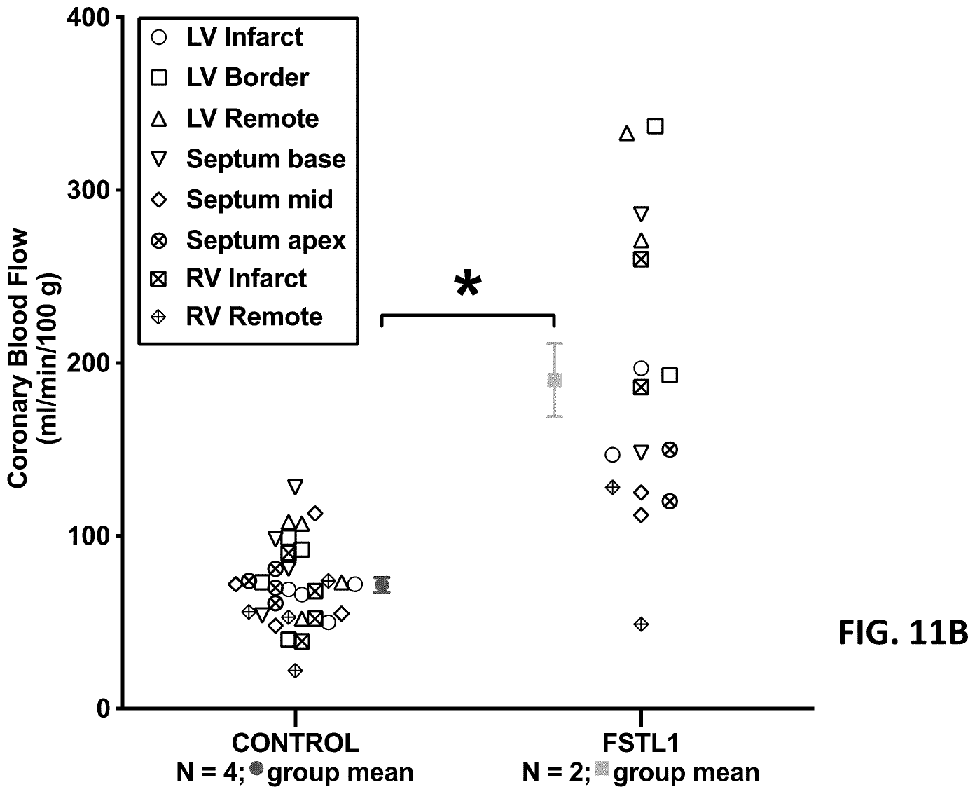

In an embodiment, blood flow to the heart and skeletal muscle can be increased following the FSTL1 treatment of the present disclosure. For example, FIG. 11A illustrates the effect of FSTL1 treatment on skeletal muscle blood flow. FIG. 11B illustrates the effect of FSTL1 treatment on coronary blood flow. FSTL-1 can attenuate the loss of functional capacity in both coronary and cerebral arterioles, demonstrating its potential to improve vascular function in an experimental setting of cardiometabolic HFrEF. Furthermore, FSTL1 can improve coronary and/or peripheral vascular function.

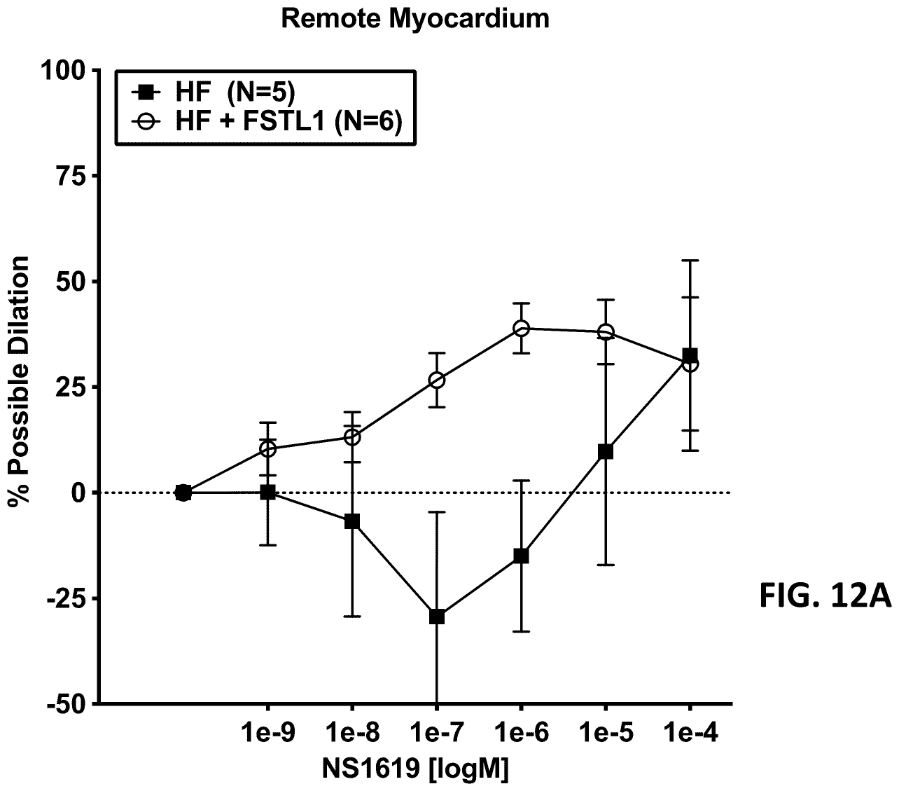

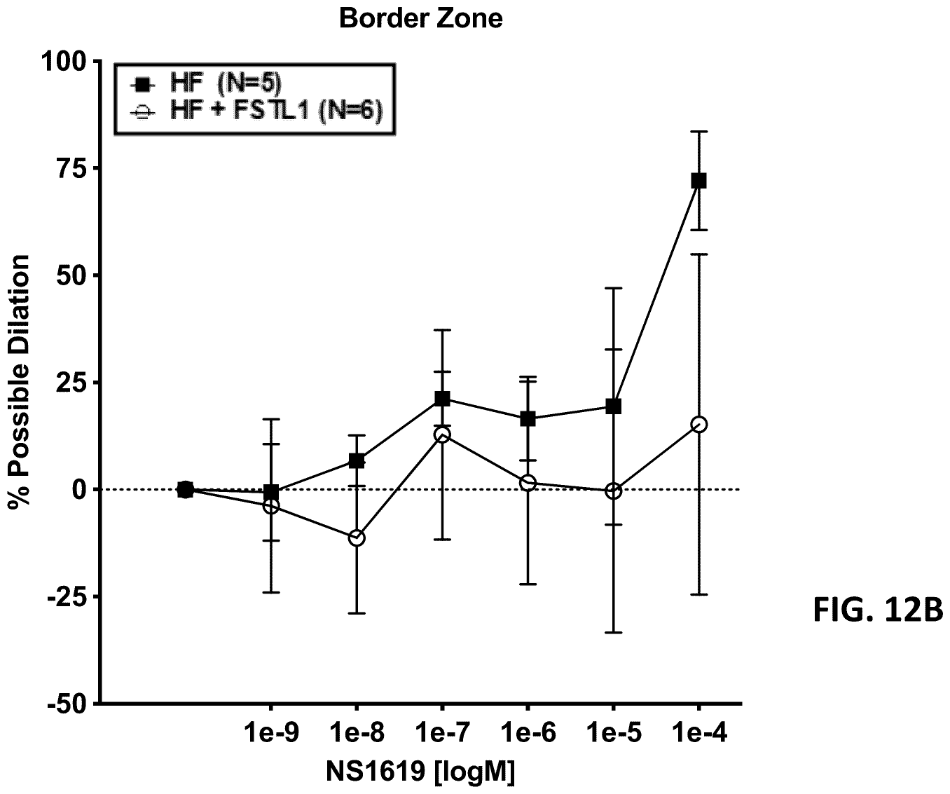

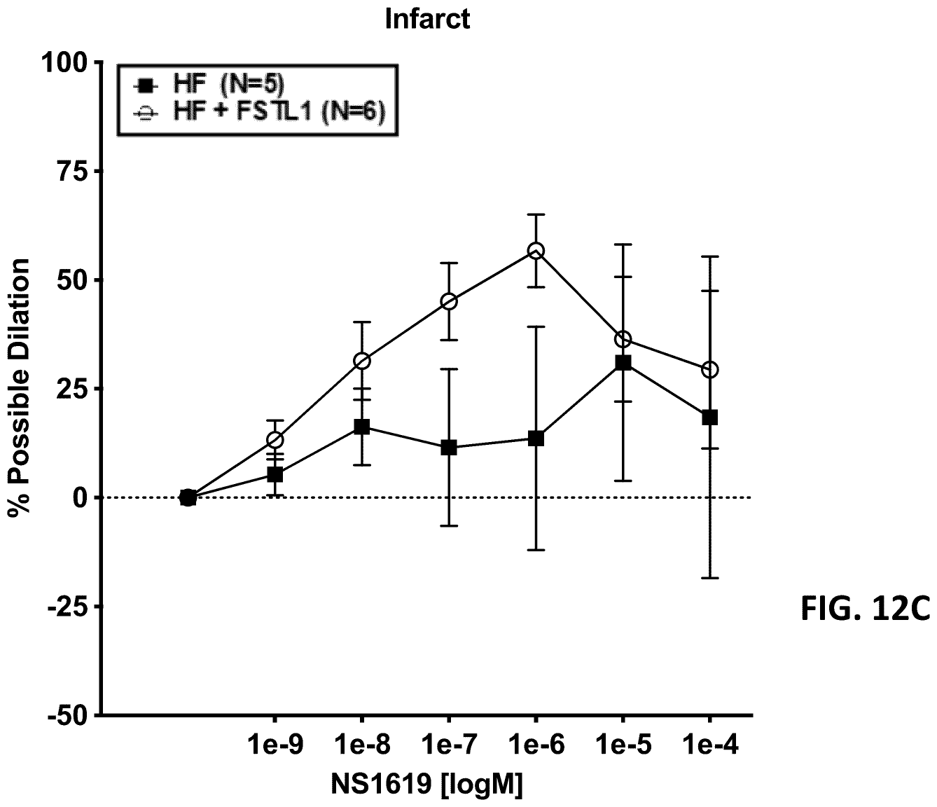

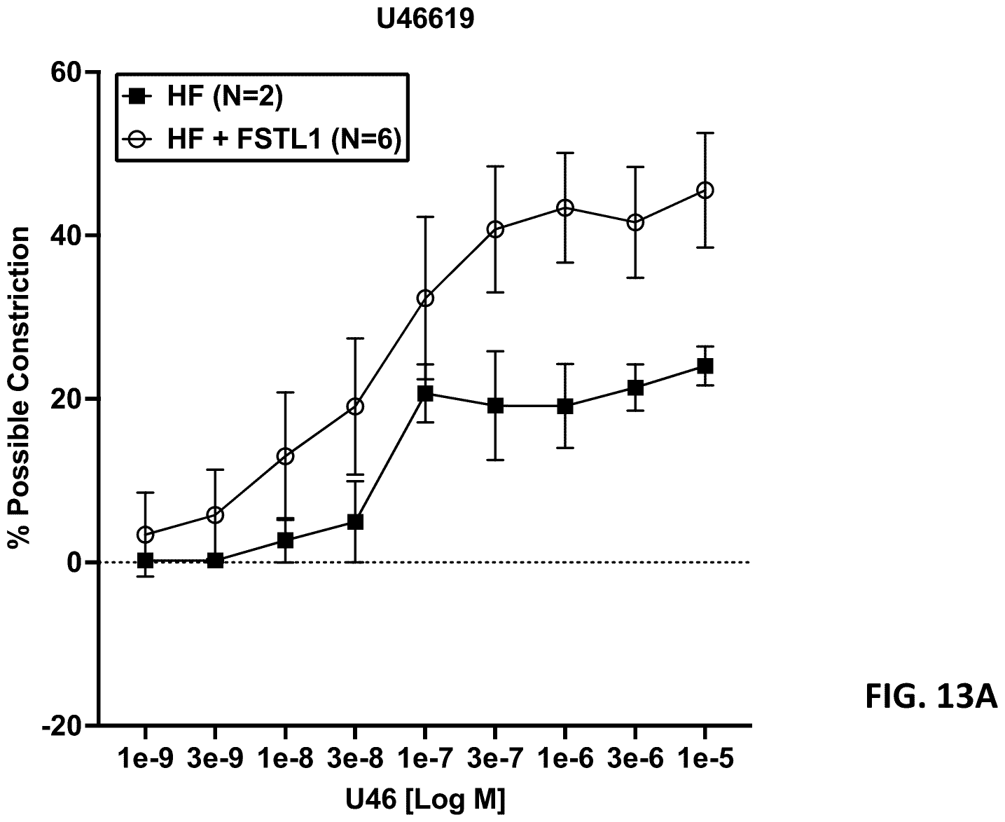

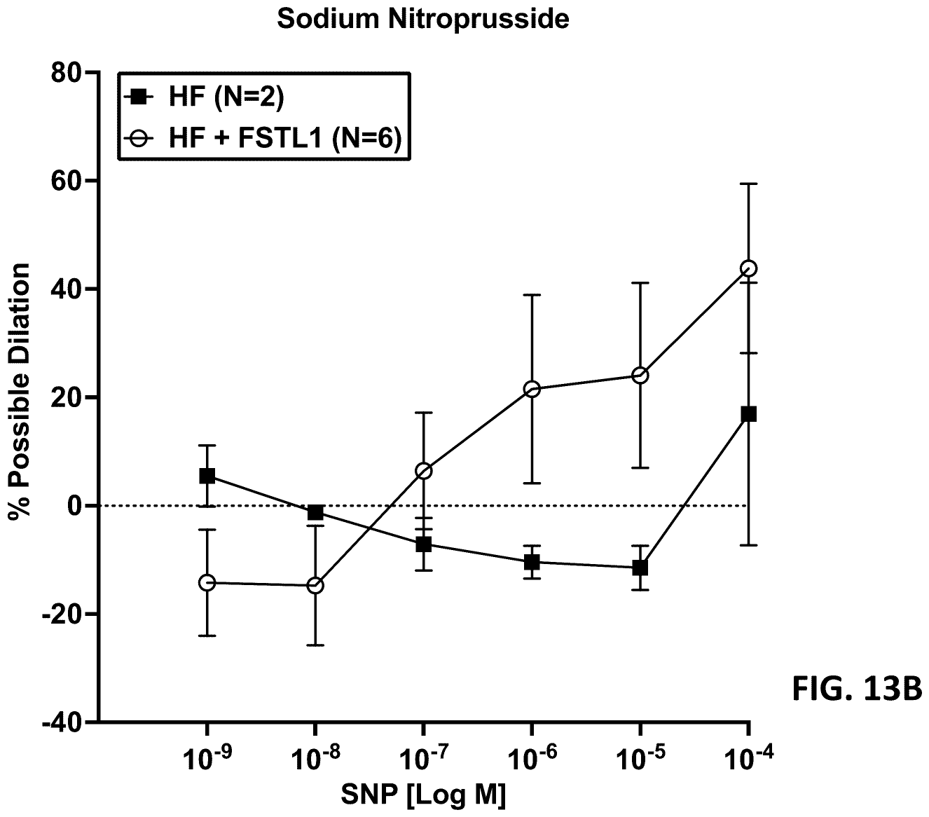

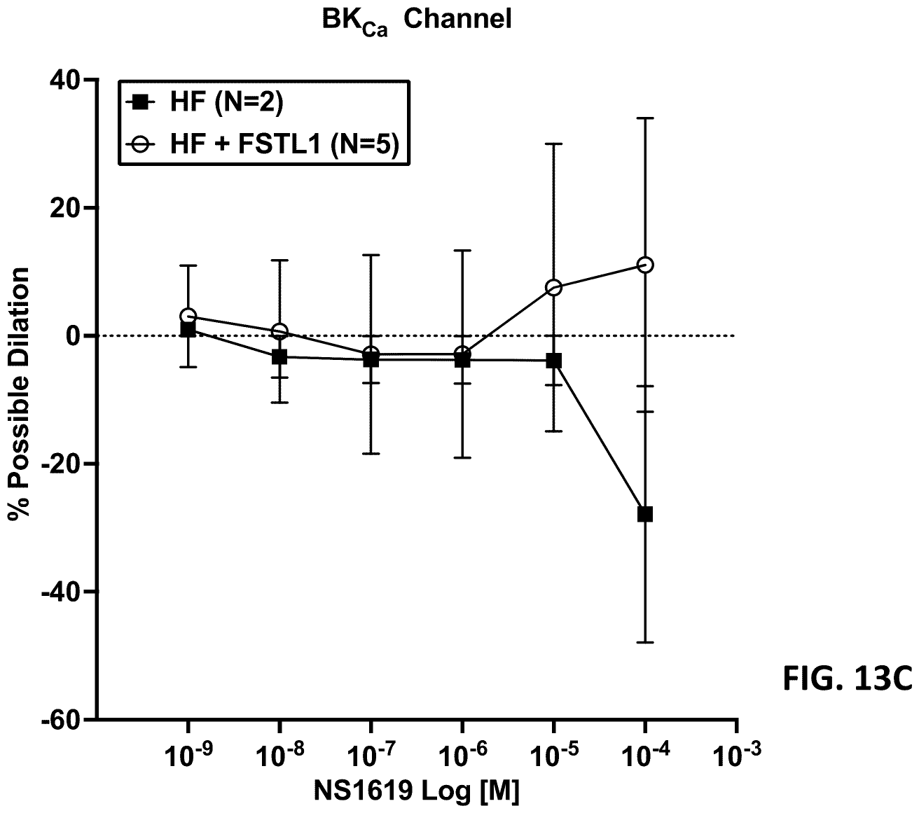

In an embodiment, the treatment with the FSTL1 of the present disclosure can attenuate the loss of TXA2-mediated vasoconstriction. For example, NS-1619-induced vasodilatory capacity can be increased in MI+FSTL-1 animals compared to MI in the remote region of the heart, but not the infarct nor border regions (FIGS. 12A-12C). In cerebral arterioles, the vasoconstrictive response to U46619 can be also dependent on experimental group (interaction), with increased constrictive capacity observed in MI+FSTL-1 compared to MI animals (FIGS. 13A-13C).

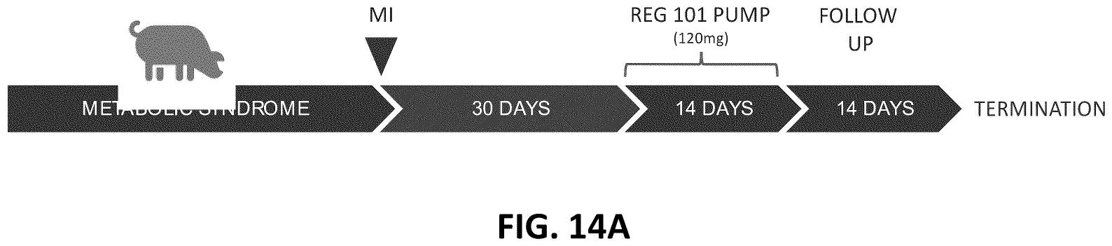

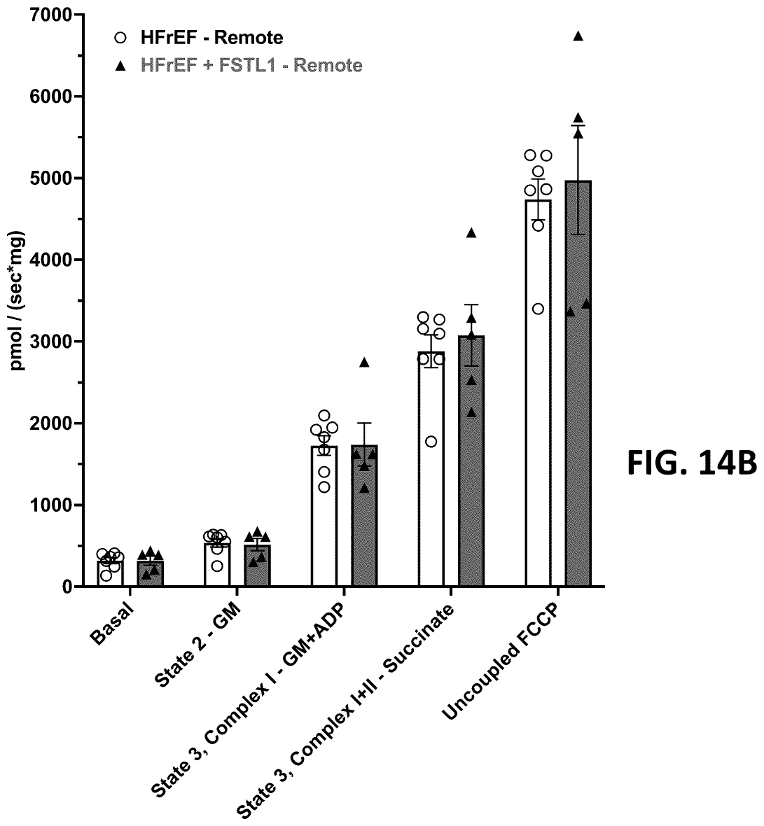

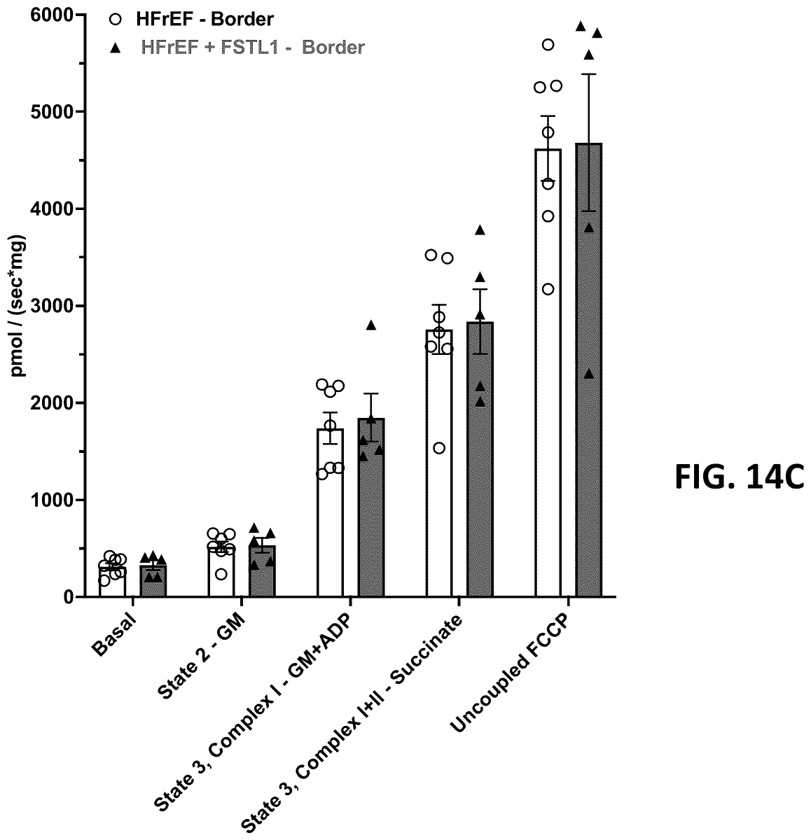

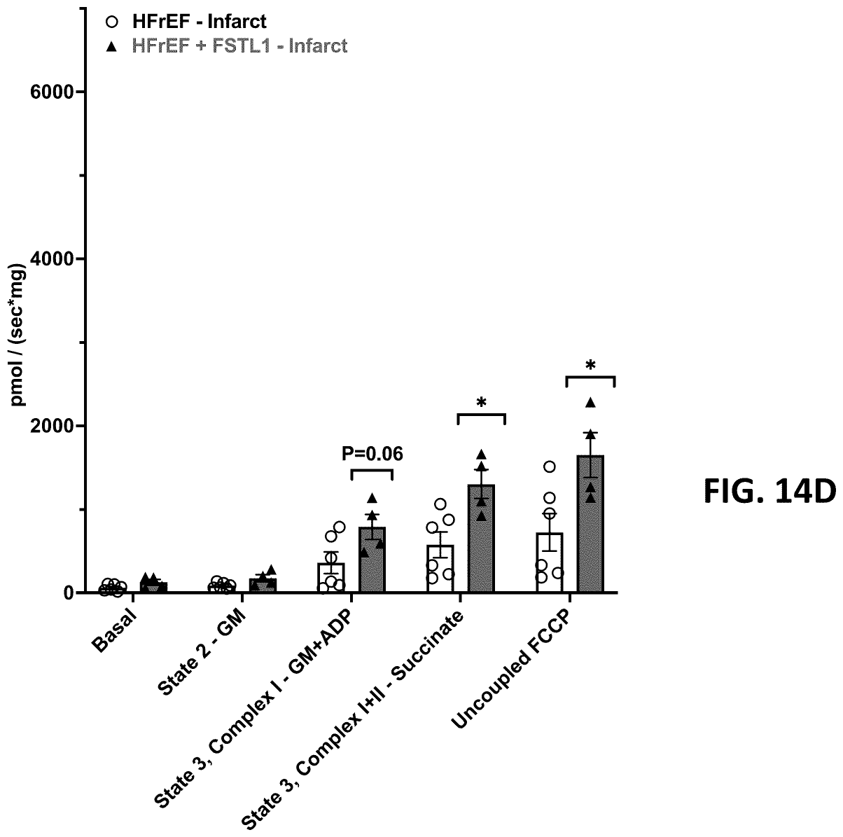

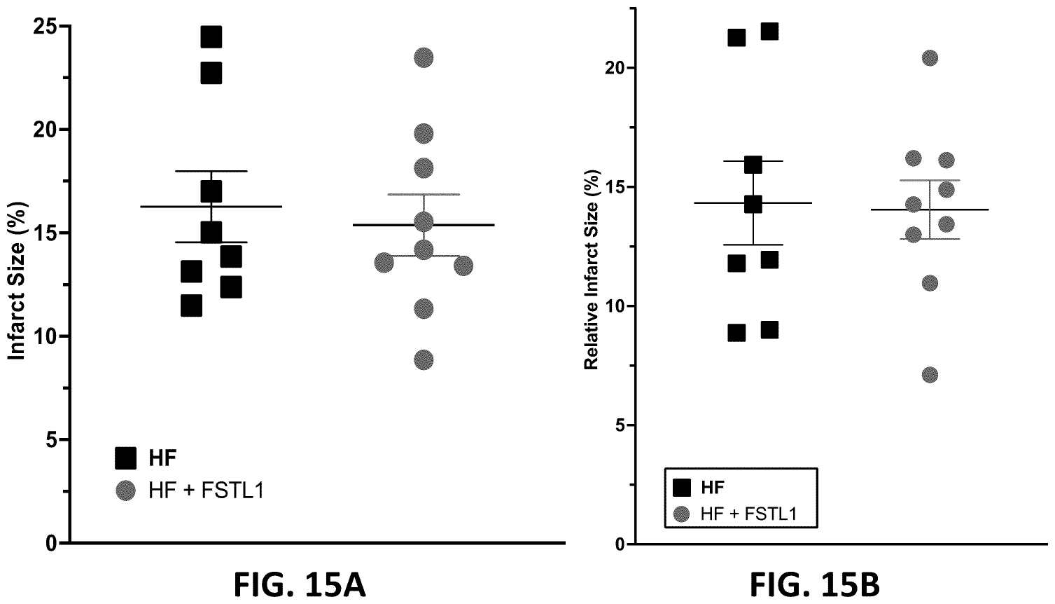

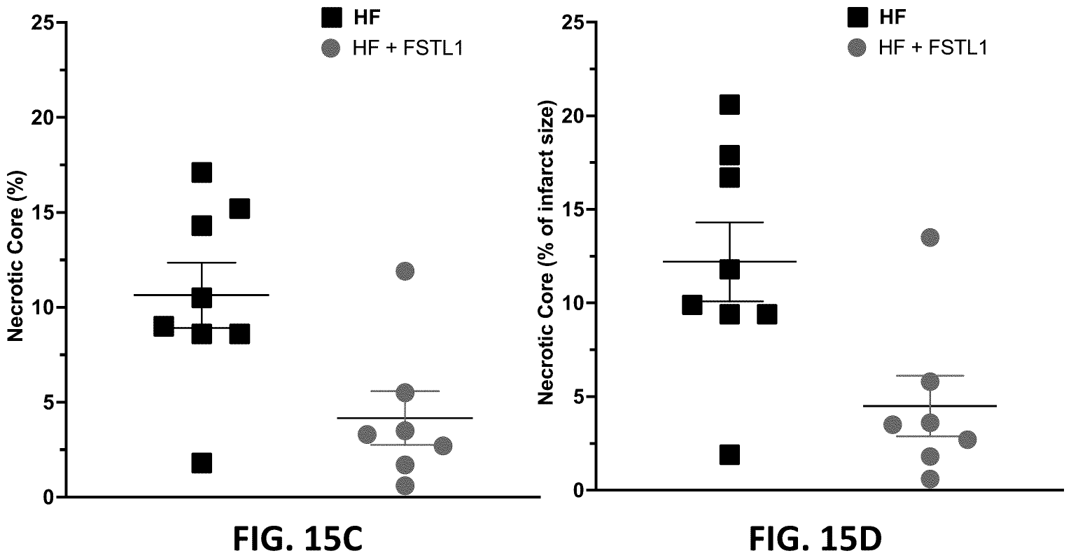

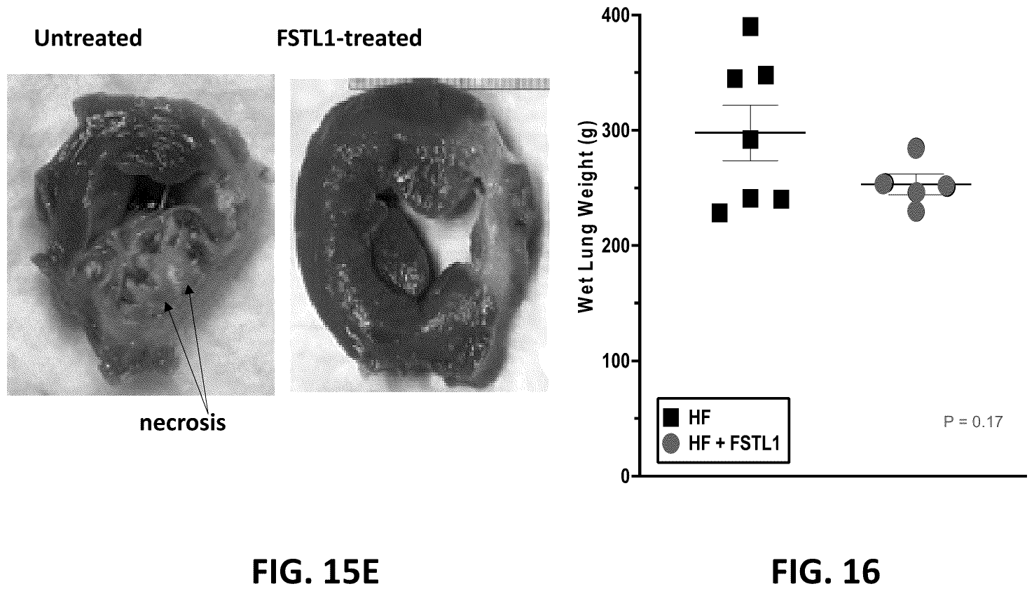

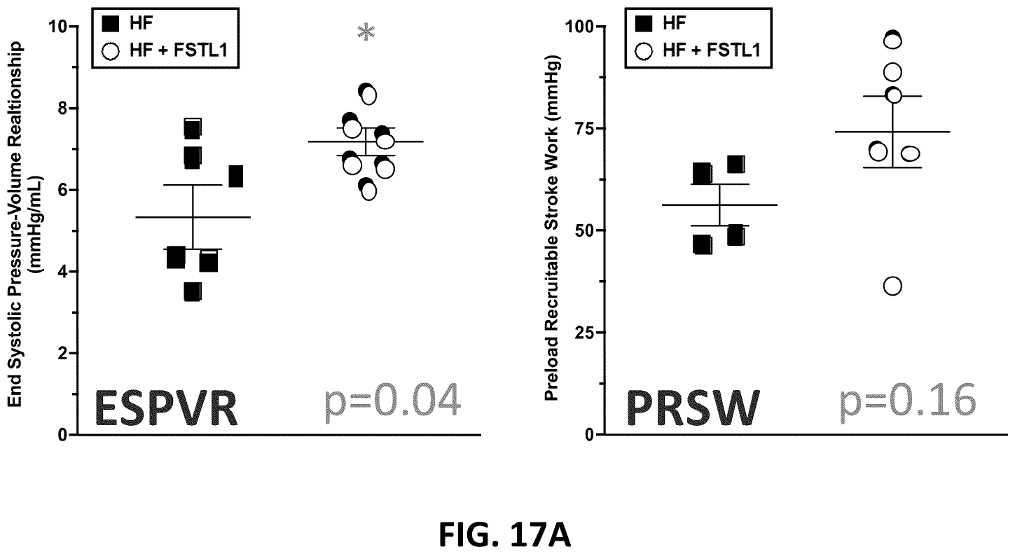

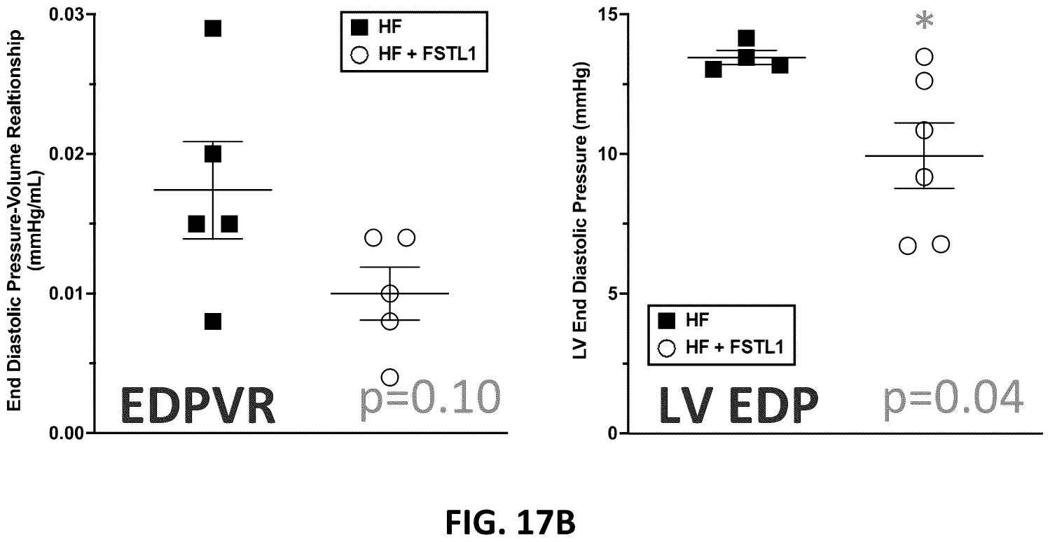

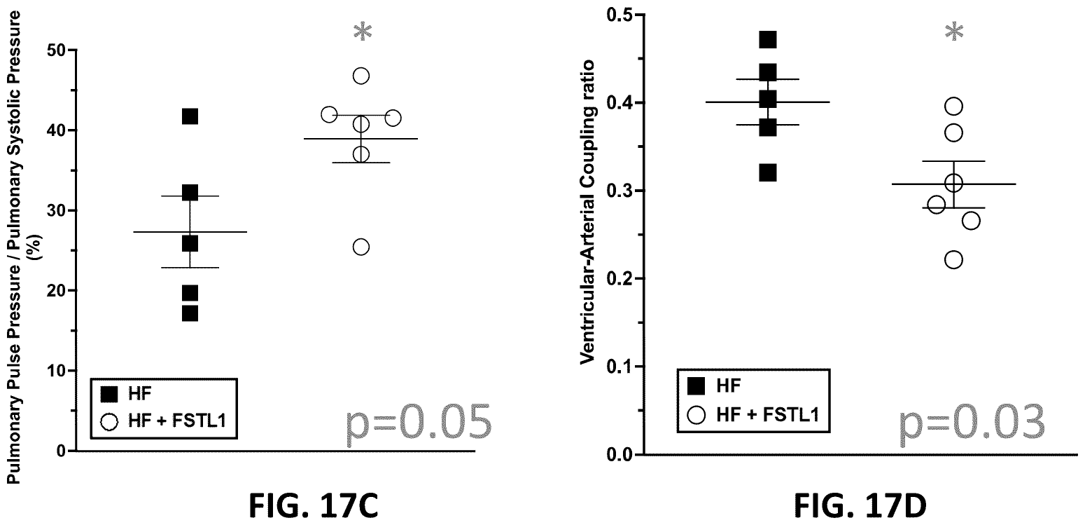

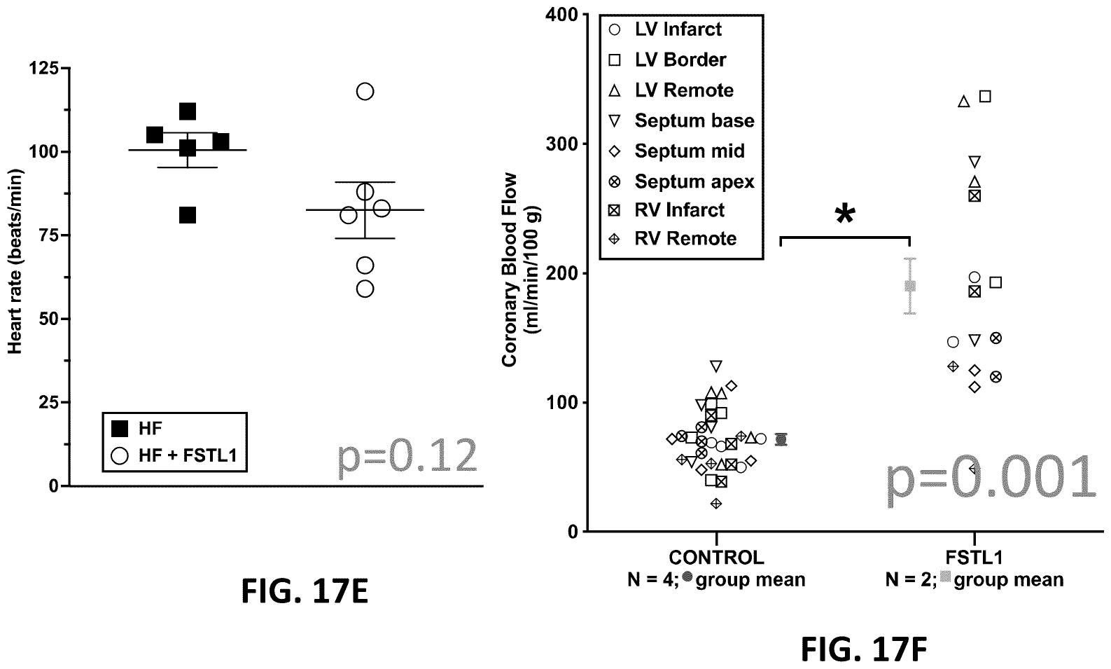

In an embodiment, the treatment with the FSTL1 of the present disclosure can improve mitochondrial function in the infarct area (FIGS. 14B-14D). In an embodiment, the treatment with the FSTL1 of the present disclosure can reduce necrosis in the infarct area (FIGS. 15C-15D). In an embodiment, the treatment with the FSTL1 of the present disclosure can reduce lung weight in myocardial-infarcted diabetic pigs (FIG. 16). In an embodiment, the treatment with the FSTL1 of the present disclosure can improve systolic function (FIG. 17A), can improve diastolic function (FIG. 17B), can restore PAPPP (FIG. 17C), can improve ventricular-arterial coupling (FIG. 17D), can reduce heart rate (FIG. 17E), and can improve coronary blood flow.

### Pharmaceutical Compositions/Kits

The presently described disclosure contemplates FSTL1 incorporated into a pharmaceutical composition (e.g., a sterile pharmaceutical composition) containing one or more pharmaceutically acceptable carriers. As used herein, a “pharmaceutically acceptable carrier” or a “pharmaceutically acceptable excipient” according to the present disclosure can be a component such as a carrier, diluent, or excipient of a composition that can be compatible with the other ingredients of the composition in that it can be combined with the agents and/or compositions of the present invention without eliminating the biological activity of the agents or the compositions (for example, FSTL1, such as non-glycosylated FSTL1), and can be suitable for use in subjects as provided herein without undue adverse side effects (such as toxicity, irritation, allergic response, and death). Side effects can be “undue” when their risk outweighs the benefit provided by the pharmaceutical composition. Non-limiting examples of pharmaceutically acceptable components include, without limitation, any of the standard pharmaceutical carriers such as phosphate buffered saline solutions, water, sterile water, polyethylene glycol, polyvinyl pyrrolidone, lecithin, arachis oil, sesame oil, emulsions such as oil/water emulsions or water/oil emulsions, microemulsions, nanocarriers and various types of wetting agents. Additives such as water, alcohols, oils, glycols, preservatives, flavoring agents, coloring agents, suspending agents, and the like can also be included in the composition along with the carrier, diluent, or excipient. In one embodiment, a pharmaceutically acceptable carrier appropriate for use in the compositions disclosed herein can be sterile, pathogen free, and/or otherwise safe for administration to a subject without risk of associated infection and other undue adverse side effects.

Any of the FSTL1-containing (such as non-glycosylated FSTL1-containing) pharmaceutical compositions disclosed herein can be formulated for administration using any number of administrative methods available in the art. Administration can be by a variety of routes including pump, patch, catheter, stent, oral, rectal, transdermal, subcutaneous, intravenous, intramuscular, intranasal, and the like. In some embodiments, the above methods of administration can be used for delivery of suspensions comprising FSTL1 (e.g., non-glycosylated FSTL1 mixed with gel or gelfoam particles). These compositions can be effective as both injectable and oral compositions. Such compositions can be prepared in a manner well known in the pharmaceutical art and comprise at least one active compound. When employed as oral compositions, the polypeptide compositions can be protected from acid digestion in the stomach by a pharmaceutically acceptable protectant.

In some embodiments, any of the FSTL1-containing (such as non-glycosylated FSTL1-containing) pharmaceutical compositions disclosed herein can be incorporated into an engineered patch for administration directly to the epicardium or damaged tissue of the myocardium. In some embodiments, highly hydrated collagen gels can be compressed in order to remove excess water and produce a dense biomaterial with improved biological and mechanical properties.

In some embodiments, any of the FSTL1-containing (such as non-glycosylated FSTL1-containing) pharmaceutical compositions disclosed herein can be delivered via an osmotic pump. In some embodiments, any of the FSTL1-containing (such as non-glycosylated FSTL1-containing) pharmaceutical compositions disclosed herein can be delivered as a single or several subcutaneous bolus.

Also provided herein are kits comprising (i) an epicardial-derived paracrine factor (such as a non-glycosylated FSTL1 polypeptide); and (ii) one or more pharmaceutically acceptable excipients. One or both of these kit components can be made to be sterile so that it can be administered to an individual in need (e.g., an individual with cardiac injury, such as MI). The kits can optionally contain a 3D collagen patch (such as any of these disclosed herein) that can be seeded or infused with the epicardial-derived paracrine factor prior to administration to a subject. Alternatively, a pre-seeded or pre-infused 3D collagen patch can be included in the kit along with written instructions regarding its use and application to injured cardiac (e.g., myocardial) tissue or the epicardium of a subject in need thereof. The kit can further comprise means for adhering the 3D collagen patch to the epicardium or to injured cardiac (e.g., myocardial) tissue such as, without limitation, suturing material.

Any of the kits disclosed herein can also include a hydrogel (such as a self-polymerizing hydrogel) as a carrier for an epicardial-derived paracrine factor (such as a hypoglycosylated FSTL1 polypeptide). In one embodiment, the kits also include one or more catheters for delivery of the hydrogel (such as a hydrogel infused with a hypoglycosylated FSTL1 polypeptide) to the endocardium, epicardium, and/or one or more damaged areas of the myocardium.

The kit can also include written instructions for using the kit, such as instructions for infusing an epicardial-derived paracrine factor into a 3D collagen patch, suturing the patch to the myocardium or epicardium, infusing an epicardial-derived paracrine factor into a hydrogel (such as a self-polymerizing hydrogel) as well as delivery of the hydrogel to the epicardium or one or more damaged areas of the myocardium via catheter technology.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### EXAMPLES

These examples are provided for illustrative purposes only and not to limit the scope of the claims provided herein.

Example 1. Generation of Ossabaw Swine with Cardiometabolic Heart Failure with Reduced Ejection Fraction and General Experimental Protocols

Following experimental protocols were applied in all of subsequent Examples provided herein.

### Generation of Cardiometabolic Heart Failure Swine Model

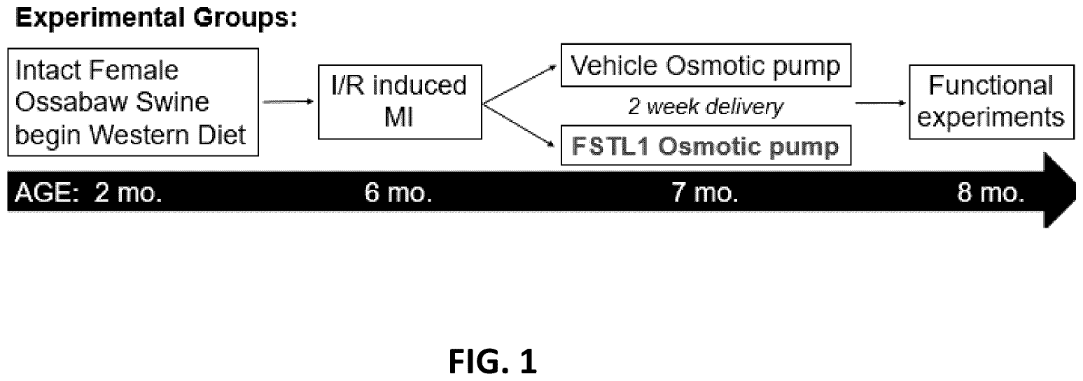

Intact female Ossabaw swine (2 mo. old) were fed a Western Diet for 4 months to develop metabolic syndrome. At 6 months of age, animals were subjected to 90 minutes ischemia followed by reperfusion (I/R) to induce MI. One month post-MI, ALZET osmotic pumps were implanted and either vehicle (MI group) or FSTL1 (MI+FSTL1 group) was delivered over two weeks, with terminal in vitro vascular experiments performed 2 month post-MI (FIG. 1).

### Pre-Surgical Preparation

Sedation: I/R MI—Telazol (6-7 mg/kg IM) and buprenorphine (0.02 to 0.05 mg/kg).

1. Ophthalmic was ointment applied to eyes to prevent corneal dryness and ulceration, followed by IM injection of clindamycin (if cefazolin is unavailable) or Excede depending on the procedure.

2. Baseline heart rate, respiratory rate, and body temperature were obtained.

3. Incision site(s) were shaved (electric clipper), scrubbed with chlorhexidine until debris was absent, and rinsed with rubbing alcohol sponges.

4. An ear catheter was placed for delivery of IV fluids and drugs throughout the procedure. If the animal was not adequately sedated to place an ear catheter, a nose cone was placed over the snout to deliver 100% oxygen mixed with 3-5% isoflurane until the animal settled.

5. A propofol bolus was administered for anesthesia induction.

6. The animal was intubated, placed on the fluoroscopy table and ventilated at a rate of 8-12 breaths per minute with a tidal volume of 10-20 mL/kg and 20-25 cm H₂O pressure. Anesthesia method-see below.

7. Patient monitoring leads were attached, and cefazolin slowly given IV (if clindamycin or Excede was not given during prep).

8. Intra-operative heat was maintained by a warm water circulating jacket placed underneath the pig. The incision site was prepared using a 3-scrub chlorhexidine regimen followed by a 70% alcohol rinse. Lastly, the surgical area was sterilely draped.

Anesthesia: I/R MI—Constant rate infusion (CRI) propofol began at approximately 10 mL/kg/hr determined by palpebral response and/or pedal withdrawal reflex.

### Survival Surgeries

Animals were maintained under a surgical plane of anesthesia using either isoflurane or propofol and ventilated with 100% oxygen. The room Circulator, trained in survival surgery procedures, monitored adequate depth of anesthesia by means of jaw tone, pedal, and palpebral reflexes at least every 15 minutes throughout the procedure. EKG, heart rate, blood pressure, SpO₂, end tidal CO₂, respiratory rate, and body temperature were continuously monitored by a member of the surgical team and/or the Circulator. If the pig's vital signs indicated the need for emergency treatment, the following drugs were available: epinephrine, lidocaine, phenylephrine (IV drip), and atropine. If the EKG indicated ventricular fibrillation or cardiac arrest, CPR was immediately administered. Defibrillation was biphasic at 200 joules. CPR, defibrillation, and drug administration was used to assist in resuscitation if necessary.

### Surgery: Surgical Procedure

Survival Angioplasty I/R MI: Following femoral artery access, a standard 6F-guide catheter was attached to a manifold assembly to allow continuous central pressure monitoring, saline flush, and Visipaque contrast injection. Under fluoroscopy guidance, an intracoronary balloon catheter was advanced into the left anterior descending (LAD) coronary artery and inflated for 90 minutes to induce ischemia followed by reperfusion. 1,000 mL saline solution mixed with amiodarone and potassium acetate was given IV CRI at 300 mL/hr for duration of I/R.

### Follistatin (FSTL1) Delivery Via Alzet Pump

Placement of ALZET Osmotic Pumps (2-4): Two±4-6 cm incisions were made on both the right and left lateral sides of the neck for the placement of the FSTL1 and EdU pump(s). A subcutaneous pocket was created by blunt dissection and the pumps (approximately 5×1.5 cm) implanted. Dead space was adequately closed with dissolvable subcutaneous sutures. The skin was closed with 2-0 Ethilon in a simple interrupted pattern with suture removal to follow in 7-10 days. Approximately 30 days post-osmotic pump implantation, non-survival intercostal thoracotomy procedures were the same as above.

### Post Anesthetic Recovery

Animals were recovered in a clean pen and kept warm with blankets and a heat lamp if needed. While recumbent and intubated the animal was continuously monitored, capillary refill time, heart and respiratory rates, and body temperature were evaluated to ensure adequate recovery. Animals were extubated once the gag reflex had returned. While semi-conscious and not sternally recumbent, the animal was monitored not less than every 15 minutes. When consciousness had returned and the animal was sternally recumbent but cannot stand, not less than once every 6 hours. Once standing and moving about but not eating/drinking normally, twice daily. When active and alert, eating/drinking normally, the animal was monitored daily for 3 days if a minor procedure or 7-10 days until sutures are removed.

### Surgery: Post-Operative Care

Animals were recovered in a clean pen and kept warm with blankets and a heat lamp if needed. While recumbent and intubated (i.e., semi-conscious) the animal was continuously monitored. Capillary refill time, heart and respiratory rates, and body temperature were evaluated to ensure adequate recovery. Animals were extubated once the gag reflex had returned. When sternal recumbency was maintained, swine was monitored every 15 minutes until alert and have regained their motor functions. Post-surgical recovery was considered complete at this time if there was no surgically related complications. Animal activity/temperament, food intake, and incisions were monitored on a daily basis post-surgery until sutures were removed.

### Surgery: Special Needs

Special needs of the animals following surgery: All animals were individually housed following surgical procedures to prevent injury or discomfort. For the first 5-7 days, all animals were kept in raised deck pens to help prevent infection.

Example 2. Non-Glycosylated FSTL1 Prevents Coronary and Cerebral Vascular Dysfunction in Ossabaw Swine with Cardiometabolic Heart Failure with Reduced Ejection Fraction

Follistatin-like 1 (FSTL1) is a glycoprotein that, when non-glycosylated or hypoglycosylated, displays regenerative properties including pro-angiogenic effects and prevention of abnormal vascular remodeling. Therefore, the objective of the current study was to assess the therapeutic benefit of human recombinant non-glycosylated FSTL1 protein on both coronary and cerebral vascular function in a pre-clinical Ossabaw swine model of myocardial infarction (MI). FSTL1 would attenuate the development of coronary and cerebral vascular dysfunction in an experimental setting of cardiometabolic heart failure with reduced ejection fraction (HFrEF).

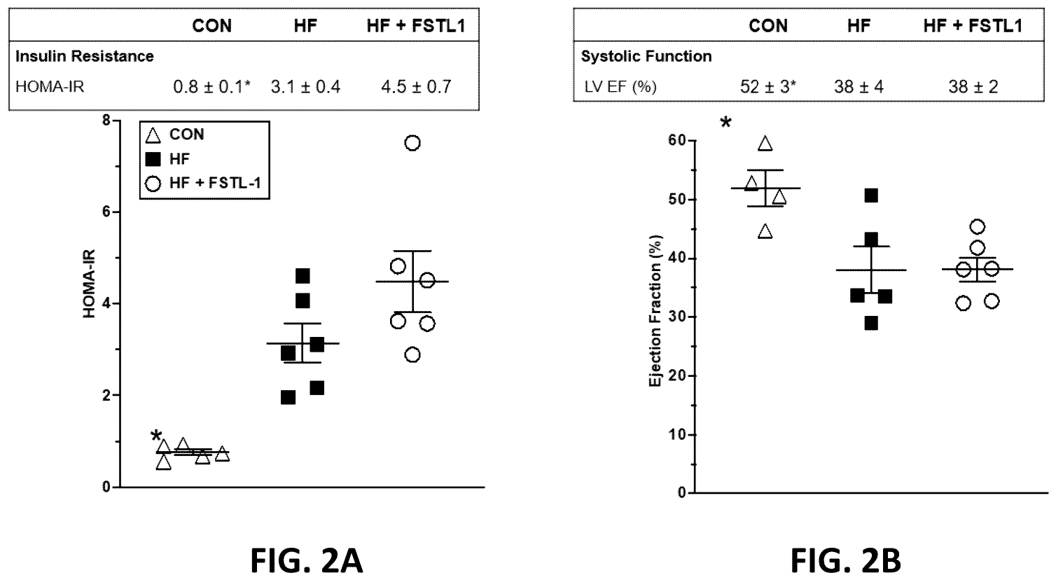

In vitro assessment of isolated coronary (n=5-6 for infarct, border, and remote regions of the left ventricle; 139.5±4.9 μm) and cerebral (n=2-6 for middle cerebral artery second order pial; 385.3±18.7 μm) arteriole function was examined using pressure myography following dose response (1e-9 to 1e-4) curves: (1) U46619 (thromboxane A2 agonist); and (2)NS-1619 (large-conductance calcium-activated potassium channel activator; BKCa). A 2×2 ANOVA (Group X Dose) was used to determine significance at the p<0.05 level (FIGS. 12A-12C; FIGS. 13A-13C). Cardiometabolic HFrEF was indicated by a combined ejection fraction of 38±2% and HOMA-IR of 3.1±0.4 (vs. 0.8±0.1 historical control) (FIGS. 2A-2B). The coronary arteriole vasodilatory response to NS-1619 was dependent on experimental group (interaction). Specifically, NS-1619-induced vasodilatory capacity was increased in MI+FSTL-1 animals compared to MI in the remote region of the heart, but not the infarct nor border regions (FIGS. 12A-12C). In cerebral arterioles, the vasoconstrictive response to U46619 was also dependent on experimental group (interaction), with increased constrictive capacity observed in MI+FSTL-1 compared to MI animals (FIGS. 13A-13C).

In addition, blood flow to the heart and skeletal muscle is increased following FSTL1 treatment (FIGS. 11A-11B). FIG. 11A illustrates the effect of FSTL1 treatment on skeletal muscle blood flow. FIG. 11B illustrates the effect of FSTL1 treatment on coronary blood flow.

In conclusion, FSTL-1 attenuates the loss of functional capacity in both coronary and cerebral arterioles, demonstrating its potential to improve vascular function in an experimental setting of cardiometabolic HFrEF. Furthermore, FSTL1 improves coronary and/or peripheral vascular function.

### Example 3. Non-Glycosylated FSTL1 Increases Probability of Survival and Reduces Infarct Size

This Example describes the results of experiments performed to investigate the effect of treatment with non-glycosylated FSTL1 on size of infarct and on probability of survival in Ossabaw Swine with cardiometabolic heart failure with reduced ejection fraction. The effect of treatment with non-glycosylated FSTL1 on size of infarct is shown in FIGS. 4A-4B. The effect of treatment with non-glycosylated FSTL1 on survival probability is shown in FIG. 3. In conclusion, treatment with non-glycosylated FSTL1 increases probability of survival and reduces infarct size in Ossabaw swine with cardiometabolic heart failure with reduced ejection fraction.

### Example 4. Non-Glycosylated FSTL1 Improves Cardiac Function

This Example describes the results of experiments performed to investigate the effect of treatment with non-glycosylated FSTL1 on cardiac function in Ossabaw Swine with cardiometabolic heart failure with reduced ejection fraction. FIGS. 5A-5B collectively illustrate that heart rate can be reduced following FSTL1 treatment. FIG. 6 illustrates that FSTL1 treatment does not alter left ventricular end systolic or diastolic volume. FIGS. 7A-7C collectively illustrate that FSTL1 prevents left ventricular diastolic dysfunction. In conclusion, treatment with non-glycosylated FSTL1 improves cardiac function.

### Example 5. Non-Glycosylated FSTL1 Improves Ventricular-Vascular Interactions

This Example describes the results of experiments performed to investigate the effect of treatment with non-glycosylated FSTL1 on ventricular-vascular interactions in Ossabaw Swine with cardiometabolic heart failure with reduced ejection fraction. FIG. 8 illustrates that pulmonary congestion is decreased following FSTL1 treatment. FIGS. 9A-9C collectively illustrate that FSTL1 prevents mismatch between ventricular function arterial load as a function of decreased contractility. FIG. 9A illustrates the effect of FSTL1 treatment on ventricular-arterial coupling ratio. FIG. 9B illustrates the effect of FSTL1 treatment on end systolic pressure-volume relationship. FIG. 9C illustrates the effect of FSTL1 treatment on preload recruitable stroke work. FIGS. 10A-10B collectively illustrate that FSTL1 prevents the narrowing of pulse pressure. FIG. 10A illustrates the effect of FSTL1 treatment on pulmonary artery pressure. FIG. 10B illustrates the effect of FSTL1 treatment on pulmonary pulse pressure/pulmonary systolic pressure. Data obtained by P-V loops were recorded under conditions of reducing preload achieved through transient occlusion of the inferior vena cava via inflation of the balloon catheter. In conclusion, non-glycosylated FSTL1 improves ventricular-vascular interactions in Ossabaw Swine with cardiometabolic heart failure with reduced ejection fraction.

### Example 6. Non-Glycosylated FSTL1 Improves Mitochondrial Function

This Example describes the results of experiments performed to investigate the effect of treatment with non-glycosylated FSTL1 on mitochondrial function in Ossabaw Swine with cardiometabolic heart failure with reduced ejection fraction.

Left ventricle (500 mg) was placed in ice-cold Buffer A (100 mM KCl, 50 mM MOPS, 5 mM MgSO4, 1 mM EGTA, 1 mM ATP). The tissue was then minced and transferred to a glass homogenization tube and homogenized with a teflon pestle. One volume of mitochondrial isolation buffer (220 mM Mannitol, 70 mM Sucrose, 10 mM Tris-base, 1 mM EDTA) supplemented with 0.5 mg/mL protease was added to the sample and inverted slowly. The samples were centrifuged at 800 g for 10 minutes at 4° C. The supernatant was transferred to a new tube, and one volume of mitochondrial isolation buffer supplemented with protease inhibitors was added to the supernatant to inactivate the protease. The supernatant was centrifuged at 10,000 g for 10 minutes at 4° C. The supernatant was discarded and the pellet resuspended in mitochondrial isolation buffer containing 0.1% BSA and centrifuged at 10,000 g for 10 minutes at 4° C. The supernatant was discarded and the pellet was resuspended in mitochondrial preservation buffer (0.5 mM EGTA, 3 mM MgCl₂·6H₂O, 60 mM K-lactobionate, 20 mM Taurine, 10 mM KH2P04, 20 mM HEPES, 110 mM Sucrose, 1 g/l BSA, 20 mM Histidine, 20 μM vitamin E succinate, 3 mM glutathione, 1 μM leupeptine, 2 mM glutamate, 2 mM malate, 2 mM Mg-ATP).

Mitochondrial respiration was assessed using high-resolution respirometry (Oroboros Oxygraph-2k; Oroboros Instruments; Innsbruck, Austria). Briefly, oxygen flux was measured by addition of glutamate (5 mM) and malate (2 mM) to the chambers in the absence of ADP (State 2-GM) for assessment of State 2 respiration. Oxidative phosphorylation (OXPHOS) with electron flux through complex I was then quantified by titration of ADP (25-125 μM) (GM+ADP: State 3-Complex I) for assessment of State 3 respiration. Maximal ADP respiration with electron flux through both complex I and complex II was assessed by the addition of succinate (10 mM) (Succinate: State 3-Complex I+II). Finally, maximal capacity of the electron transport system was assessed by uncoupling with the addition of FCCP (Carbonyl cyanide 4-(trifluoromethoxy)phenylhydrazone, 0.25 μM) (Uncoupled).

Four states were analyzed (State 2 GM, State 3 complex 1 GM+ADP, State 3 complex I+II Succinate, and uncoupled FCCP). As shown in FIGS. 14B-14D, oxygen flow was not affected by FSTL1 treatment in the remote portions of the heart, the areas not directed affected by the infarct (FIG. 14B). Similarly, there were no differences of oxygen flow in the injury border zone at the four oxidative states measured (FIG. 14C). In contrast, in the infarct zone, oxygen flow (although generally reduced) was significantly increased by FSTL1 treatment (FIG. 14D). In conclusion, FSTL1 SubQ treatment of myocardial infarcted diabetic pigs (using osmotic pump 120 mg/14 days) specifically improved mitochondrial function in infarct area.

### Example 7. Non-Glycosylated FSTL1 Reduces Necrosis in Infarct Area

This Example describes the results of experiments performed to investigate the effect of treatment with non-glycosylated FSTL1 on necrosis in Ossabaw Swine with cardiometabolic heart failure with reduced ejection fraction.

The left ventricle was sectioned from apex to base into five transverse slices. Pictures of the apical and basal face of each slice were taken, and ImageJ software was used to analyze the digital images. For each slice, left ventricle and infarct areas were manually traced from photographs of the apical and basal sides. The infarct was calculated as a percentage of the left ventricle for each slice, and the results of both the apical and basal sides were averaged and multiplied by the weight of each individual slice weight to calculate infarct size. The infarct size from all slices was summed and divided by the sum of the left ventricle.

Evaluation of infarct size showed no significant differences of infarct size among treatments, indicating consistency in the performance of surgical coronary occlusion (FIGS. 15A-15B). Analysis of the tissue within the scar showed more than two-fold reduction of the necrotic area in the FSTL1-treated group (FIG. 15C), even when corrected by infarct size (FIG. 15D). In conclusion, non-glycosylated FSTL1 reduced necrosis in the infarct area.

### Example 8. Non-Glycosylated FSTL1 Reduces Lung Weight

This Example describes the results of experiments performed to investigate the effect of treatment with non-glycosylated FSTL1 on lung weight in Ossabaw Swine with cardiometabolic heart failure with reduced ejection fraction.

Direct weight of lungs of FSTL1-treated and untreated pigs was measured. FSTL1-treated animals showed statistically significant reduced lung weight compared to untreated animals, demonstrating indirect evidence of improved cardiac function (FIG. 16). In conclusion, non-glycosylated FSTL1 reduced lung weight in myocardial-infarcted diabetic pigs.

### Example 9. Non-Glycosylated FSTL1 Improves Heart Functions

This Example describes the results of experiments performed to investigate the effect of treatment with non-glycosylated FSTL1 on heart functions in Ossabaw Swine with cardiometabolic heart failure with reduced ejection fraction.

P-V loops were recorded under conditions of reducing preload achieved through transient occlusion of the inferior vena cava via inflation of the balloon catheter. Indexes of LV function were generated using a minimum of 10 consecutive cardiac cycles with Lab Scribe software (iWorx, Dover, NH), including HR, LV end-systolic and diastolic volume (LVESV and LVEDV, respectively), LV end-systolic and diastolic pressure, ejection fraction (EF %), stroke volume, stroke work (SW), rate-pressure product, and cardiac output. Other previously published indexes of LV function proposed to be less sensitive to myocardial load and/or morphology were also determined using at least 15 consecutive cardiac cycles of constantly reducing preload, including the end-systolic P-V relationship (ESPVR) and preload recruitable SW (PRSW). In conclusion, non-glycosylated FSTL1 treatment improved systolic function (FIG. 17A), improved diastolic function (FIG. 17B), restored PAPPP (FIG. 17C), improved ventricular-arterial coupling (FIG. 17D), reduced heart rate (FIG. 17E), and improved coronary blood flow.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Sequences

| SEQ | | |
|---|---|---|
| ID | | |
| NOs | Sequences | Description |
| 1 | MWKRWLALALALVAVAWVRAEEELRSKSKI | FSTL1 amino |
| | CANVFCGAGRECAVTEKGEPTCLCIEQCKP | acid |
| | HKRPVCGSNGKTYLNHCELHRDACLTGSKI | sequence |
| | QVDYDGHCKEKKSVSPSASPVVCYQSNRDE | (NCBI |
| | LRRRIIQWLEAEIIPDGWFSKGSNYSEILD | Reference |
| | KYFKNFDNGDSRLDSSEFLKFVEQNETAIN | Sequence: |
| | ITTYPDQENNKLLRGLCVDALIELSDENAD | NP_009016.1) |
| | WKLSFQEFLKCLNPSFNPPEKKCALEDETY | |
| | ADGAETEVDCNRCVCACGNWVCTAMTCDGK | |
| | NQKGAQTQTEEEMTRYVQELQKHQETAEKT | |
| | KRVSTKEI | |
| 2 | AGGAGAATGGGGAGGAGCTGGGGGGGCAGG | FSTL1 |
| | CAGGCGGGGAAGGAGAGGTCTTAAGGGGCG | nucleic |
| | GCGAGGGGAGGTCGCATTTCCTCCGAGGCT | acid |
| | GGCGATCGGCGGAGCTCCCACCTCCGCTTA | sequence |
| | CAGCTCGCTGCCGCCGTCCTGCCCCGCGCC | (NCBI |
| | CCCAGGAGACCTGGACCAGACCACGATGTG | Reference |
| | GAAACGCTGGCTCGCGCTCGCGCTCGCGCT | Sequence: |
| | GGTGGCGGTCGCCTGGGTCCGCGCCGAGGA | NM_007085.4) |
| | AGAGCTAAGGAGCAAATCCAAGATCTGTGC | |
| | CAATGTGTTTTGTGGAGCCGGCCGGGAATG | |
| | TGCAGTCACAGAGAAAGGGGAACCCACCTG | |
| | TCTCTGCATTGAGCAATGCAAACCTCACAA | |
| | GAGGCCTGTGTGTGGCAGTAATGGCAAGAC | |
| | CTACCTCAACCACTGTGAACTGCATCGAGA | |
| | TGCCTGCCTCACTGGATCCAAAATCCAGGT | |
| | TGATTACGATGGACACTGCAAAGAGAAGAA | |
| | ATCCGTAAGTCCATCTGCCAGCCCAGTTGT | |
| | TTGCTATCAGTCCAACCGTGATGAGCTCCG | |
| | ACGTCGCATCATCCAGTGGCTGGAAGCTGA | |
| | GATCATTCCAGATGGCTGGTTCTCTAAAGG | |
| | CAGCAACTACAGTGAAATCCTAGACAAGTA | |
| | TTTTAAGAACTTTGATAATGGTGATTCTCG | |
| | CCTGGACTCCAGTGAATTCCTGAAGTTTGT | |
| | GGAACAGAATGAAACTGCCATCAATATTAC | |
| | AACGTATCCAGACCAGGAGAACAACAAGTT | |
| | GCTTAGGGGACTCTGTGTTGATGCTCTCAT | |
| | TGAACTGTCTGATGAAAATGCTGATTGGAA | |
| | ACTCAGCTTCCAAGAGTTTCTCAAGTGCCT | |
| | CAACCCATCTTTCAACCCTCCTGAGAAGAA | |
| | GTGTGCCCTGGAGGATGAAACGTATGCAGA | |
| | TGGAGCTGAGACCGAGGTGGACTGTAACCG | |
| | CTGTGTCTGTGCCTGTGGAAATTGGGTCTG | |
| | TACAGCCATGACCTGTGACGGAAAGAATCA | |
| | GAAGGGGGCCCAGACCCAGACAGAGGAGGA | |
| | GATGACCAGATATGTCCAGGAGCTCCAAAA | |
| | GCATCAGGAAACAGCTGAAAAGACCAAGAG | |
| | AGTGAGCACCAAAGAGATCTAATGAGGAGG | |
| | CACAGACCAGTGTCTGGATCCCAGCATCTT | |
| | CTCCACTTCAGCGCTGAGTTCAGTATACAC | |
| | AAGTGTCTGCTACAGTCGCCAAATCACCAG | |
| | TATTTGCTTATATAGCAATGAGTTTTATTT | |
| | TGTTTATTTGTTTTGCAATAAAGGATATGA | |
| | AGGTGGCTGGCTAGGAAGGGAAGGGCCACA | |
| | GCCTTCATTTCTAGGAGTGCTTTAAGAGAA | |
| | ACTGTAAATGGTGCTCTGGGGCTGGAGGCT | |
| | AGTAAGGAAACTGCATCACGATTGAAAGAG | |
| | GAACAGACCCAAATCTGAACCTCTTTTGAG | |
| | TTTACTGCATCTGTCAGCAGGCTGCAGGGA | |
| | GTGCACACGATGCCAGAGAGAACTTAGCAG | |
| | GGTGTCCCCGGAGGAGAGGTTTGGGAAGCT | |
| | CCACGGAGAGGAACGCTCTCTGCTTCCAGC | |
| | CTCTTTCCATTGCCGTCAGCATGACAGACC | |
| | TCCAGCATCCACGCATCTCTTGGTCCCAAT | |
| | AACTGCCTCTAGATACATAGCCATACTGCT | |
| | AGTTAACCCAGTGTCCCTCAGACTTGGATG | |
| | GAGTTTCTGGGAGGGTACACCCAAATGATG | |
| | CAGATACTTGTATACTTTGAGCCCCTTAGC | |
| | GACCTAACCAAATTTTAAAAATACTTTTTA | |
| | CCAAAGGTGCTATTTCTCTGTAAAACACTT | |
| | TTTTTTGGCAAGTTGACTTTATTCTTCAAT | |
| | TATTATCATTATATTATTGTTTTTTAATAT | |
| | TTTATTTTCTTGACTAGGTATTAAGCTTTT | |
| | GTAATTATTTTTCAGTAGTCCCACCACTTC | |
| | ATAGGTGGAAGGAGTTTGGGGTTCTTCCTG | |
| | GTGCAGGGGCTGAAATAACCCAGATGCCCC | |
| | CACCCTGCCACATACTAGATGCAGCCCATA | |
| | GTTGGCCCCCCTAGCTTCCAGCAGTCCACT | |
| | ATCTGCCAGAGGAGCAAGGGTGCCTTAGAC | |
| | CGAAGCCAGGGGAAGAAGCATCTTCATAAA | |
| | AAACTTTCAAGATCCAAACATTAATTTGTT | |
| | TTTATTTATTCTGAGAAGTTGAGGCAAATC | |
| | AGTATTCCCAAGGATGGCGACAAGGGCAGC | |
| | CAAGCAGGGCTTAGGATATCCCAGCCTACC | |
| | AATATGCTCATTCGACTAACTAGGAGGGTG | |
| | AGTTGGCCCTGTCTCTTCTTTTTTCTGGAC | |
| | CTCAGTTTCCTCAGTGAGCTGGTAAGAATG | |
| | CACTAACCTTTTGATTTGATAAGTTATAAA | |
| | TTCTGTGGTTCTGATCATTGGTCCAGAGGG | |
| | GAGATAGGTTCCTGTGATTTTTCCTTCTTC | |
| | TCTATAGAATAAATGAAATCTTGTTACTAG | |
| | AACAAGAAATGTCAGATGGCCAAAAACAAG | |
| | ATGACCAGATTTGATCTCAGCCTGATGACC | |
| | CTACAGGTCGTGCTATGATATGGAGTCCTC | |
| | ATGGGTAAAGCAGGAAGAGAGTGGGAAAGA | |
| | GAACCACCCCACTCTGTCTTCATATTTGCA | |
| | TTTCATGTTTAACCTCCGGCTGGAAATAGA | |
| | AAGCATTCCCTTAGAGATGAGGATAAAAGA | |
| | AAGTTTCAGATTCAACAGGGGGAAGAAAAT | |
| | GGAGATTTAATCCTAAAACTGTGACTTGGG | |
| | GAGGTCAGTCATTTACAGTTAGTCCTGTGT | |
| | CTTTCGACTTCTGTGATTATTAACCCCACT | |
| | CACTACCCTGTTTCAGATGCATTTGGAATA | |
| | CCAAAGATTAAATCCTTGACATAAGATCTC | |
| | ATTTGCAGAAAGCAGATTAAAGACCATCAG | |
| | AAGGAAATTATTTAGGTTGTAATGCACAGG | |
| | CAACTGTGAGAAACTGTTGTGCCAAAAATA | |
| | GAATTCCTTCTAGTTTTTCTTGTTCTCATT | |
| | TGAAAGGAGAAAATTCCACTTTGTTTAGCA | |
| | TTTCAAGCTTTTATGTATCCATCCCATCTA | |
| | AAAACTCTTCAAACTCCACTTGTTCAGTCT | |
| | GAAATGCAGCTCCCTGTCCAAGTGCCTTGG | |
| | AGAACTCACAGCAGCACGCCTTAATCAAAG | |
| | GTTTTACCAGCCCTTGGACACTATGGGAGG | |
| | AGGGCAAGAGTACACCAATTTGTTAAAAGC | |
| | AAGAAACCACAGTGTCTCTTCACTAGTCAT | |
| | TTAGAACATGGTTATCATCCAAGACTACTC | |
| | TACCCTGCAACATTGAACTCCCAAGAGCAA | |
| | ATCCACATTCCTCTTGAGTTCTGCAGCTTC | |
| | TGTGTAAATAGGGCAGCTGTCGTCTATGCC | |
| | GTAGAATCACATGATCTGAGGACCATTCAT | |
| | GGAAGCTGCTAAATAGCCTAGTCTGGGGAG | |
| | TCTTCCATAAAGTTTTGCATGGAGCAAACA | |
| | AACAGGATTAAACTAGGTTTGGTTCCTTCA | |
| | GCCCTCTAAAAGCATAGGGCTTAGCCTGCA | |
| | GGCTTCCTTGGGCTTTCTCTGTGTGTGTAG | |
| | TTTTGTAAACACTATAGCATCTGTTAAGAT | |
| | CCAGTGTCCATGGAAACATTCCCACATGCC | |
| | GTGACTCTGGACTATATCAGTTTTTGGAAA | |
| | GCAGGGTTCCTCTGCCTGCTAACAAGCCCA | |
| | CGTGGACCAGTCTGAATGTCTTTCCTTTAC | |
| | ACCTATGTTTTTAAGTAGTCAAACTTCAAG | |
| | AAACAATCTAAACAAGTTTCTGTTGCATAT | |
| | GTGTTTGTGAACTTGTATTTGTATTTAGTA | |
| | GGCTTCTATATTGCATTTAACTTGTTTTTG | |
| | TAACTCCTGATTCTTCCTTTTCGGATACTA | |
| | TTGATGAATAAAGAAATTAAAGTGATGGTT | |
| | TTGGTTTCCTTTCCCCCAATTAAGGCCAAA | |
| | TAAAGTCGTGAGAACATTACCCATTTA | |

We claim:

1. A method of aiding cardiometabolic heart failure treatment, the method comprising contacting cardiac tissue with a non-glycosylated follistatin-like 1 (FSTL1) polypeptide.

2. A method of treating cardiometabolic heart failure, the method comprising contacting cardiac tissue with a non-glycosylated follistatin-like 1 (FSTL1) polypeptide.

3. A method of repairing cardiac tissue following cardiometabolic heart failure, the method comprising contacting the cardiac tissue with a non-glycosylated follistatin-like 1 (FSTL1) polypeptide.

4. The method of any one of claims 1-3, wherein the cardiometabolic heart failure is a heart failure with reduced ejection fraction (HFrEF).

5. The method of any one of claims 1-4, wherein the cardiometabolic heart failure is a diabetic cardiomyopathy.

6. The method of any one of claims 1-5, wherein the FSTL1 decreases heart failure mortality.

7. The method of any one of claims 1-5, wherein the FSTL1 attenuates the development of coronary and cerebral vascular dysfunction.

8. The method of any one of claims 1-5, wherein the FSTL1 improves cardiac function.

9. The method of claim 8, wherein the FSTL1 reduces heart rate.

10. The method of claim 8, wherein the FSTL1 does not alter left ventricular volume and systolic or diastolic volume.

11. The method of claim 8, wherein the FSTL1 prevents left ventricular diastolic dysfunction.

12. The method of any one of claims 1-5, wherein the FSTL1 improves ventricular-vascular interactions.

13. The method of claim 12, wherein the FSTL1 decreases pulmonary congestion.

14. The method of claim 12, wherein the FSTL1 prevents mismatch between ventricular function and arterial load as a function of decreased contractility.

15. The method of claim 12, wherein the FSTL1 prevents the narrowing of pulse pressure.

16. The method of any one of claims 1-5, wherein the FSTL1 improves coronary and/or peripheral vascular function.

17. The method of claim 16, wherein the FSTL1 increases blood flow to the heart and skeletal muscles.

18. The method of claim 16, wherein the FSTL1 attenuates impaired BK_{Ca} channel-mediated coronary arteriole dilatory capacity in remote coronary vessels.

19. The method of claim 16, wherein the FSTL1 attenuates the loss of TXA₂-mediated vasoconstriction.

20. The method of any one of claims 1-5, wherein the FSTL1 improves mitochondrial function.

21. The method of any one of claims 1-5, wherein the FSTL1 reduces necrosis in infarct area.

22. The method of any one of claims 1-5, wherein the FSTL1 reduces lung weight.

23. The method of any one of claims 1-22, wherein the FSTL1 is delivered by systemically.

24. The method of any one of claims 1-22, wherein the FSTL1 is delivered endocardially.

25. The method of claim 24, wherein the endocardial delivery is via a catheter.

26. The method of any one of claims 1-22, wherein the FSTL1 is delivered epicardially.

27. The method of any one of claims 1-22, wherein the FSTL1 is delivered using a drug-eluting stent.

28. The method of any one of claims 1-22, wherein the FSTL1 is delivered by a hydrogel embedded or seeded with the FSTL1.

29. The method of any one of claims 1-22, wherein the FSTL1 is delivered by a collagen patch embedded or seeded with the FSTL1.

30. The method of any one of claims 1-22, wherein the FSTL1 is delivered via an osmotic pump.

31. The method of any one of claims 1-22, wherein the FSTL1 is delivered as a single or several subcutaneous bolus.

32. The method of any one of claims 1-22, wherein the FSTL1 is delivered by a coronary infusion.

33. The method of any one of claims 1-22, wherein the FSTL1 is expressed in the heart by use of modified RNAs (modRNAs).

34. The method of any one of claims 1-22, wherein the FSTL1 is expressed by genomic editing.

35. The method of any one of claims 24-34, wherein the cardiac tissue is contacted from one or more of an epicardial site, an endocardial site, and/or through direct injection into the myocardium.

36. The method of any one of claims 33-34, the method further comprising an inhibitor of FSTL1 glycosylation.

37. The method of claim 36, wherein the inhibitor of FSTL1 glycosylation comprises tunicamycin.


SEQUENCE LISTING (2 sequences)

SEQ ID NO: 1 - protein, 308 aa, Homo sapiens
  REGION 1..308
MWKRWLALALALVAVAWVRAEEELRSKSKICANVFCGAGRECAVTEKGEPTCLCIEQCKPHKRPVCGSNGKTYLNHCELHRDACLTGSKIQVDYDGHCKEKKSVSPSASPVVCYQSNRDELRRRIIQWLEAEIIPDGWFSKGSNYSEILDKYFKNFDNGDSRLDSSEFLKFVEQNETAINITTYPDQENNKLLRGLCVDALIELSDENADWKLSFQEFLKCLNPSFNPPEKKCALEDETYADGAETEVDCNRCVCACGNWVCTAMTCDGKNQKGAQTQTEEEMTRYVQELQKHQETAEKTKRVSTKEI

SEQ ID NO: 2 - RNA, 3837 nt, Homo sapiens
  source 1..3837: mol_type = mRNA
  misc_feature 1..3837: note = FSTL1 nucleic acid sequence (NCBI Reference Sequence: NM_007085.4)
aggagaatggggaggagctgggggggcaggcaggcggggaaggagaggtcttaaggggcggcgaggggaggtcgcatttcctccgaggctggcgatcggcggagctcccacctccgcttacagctcgctgccgccgtcctgccccgcgcccccaggagacctggaccagaccacgatgtggaaacgctggctcgcgctcgcgctcgcgctggtggcggtcgcctgggtccgcgccgaggaagagctaaggagcaaatccaagatctgtgccaatgtgttttgtggagccggccgggaatgtgcagtcacagagaaaggggaacccacctgtctctgcattgagcaatgcaaacctcacaagaggcctgtgtgtggcagtaatggcaagacctacctcaaccactgtgaactgcatcgagatgcctgcctcactggatccaaaatccaggttgattacgatggacactgcaaagagaagaaatccgtaagtccatctgccagcccagttgtttgctatcagtccaaccgtgatgagctccgacgtcgcatcatccagtggctggaagctgagatcattccagatggctggttctctaaaggcagcaactacagtgaaatcctagacaagtattttaagaactttgataatggtgattctcgcctggactccagtgaattcctgaagtttgtggaacagaatgaaactgccatcaatattacaacgtatccagaccaggagaacaacaagttgcttaggggactctgtgttgatgctctcattgaactgtctgatgaaaatgctgattggaaactcagcttccaagagtttctcaagtgcctcaacccatctttcaaccctcctgagaagaagtgtgccctggaggatgaaacgtatgcagatggagctgagaccgaggtggactgtaaccgctgtgtctgtgcctgtggaaattgggtctgtacagccatgacctgtgacggaaagaatcagaagggggcccagacccagacagaggaggagatgaccagatatgtccaggagctccaaaagcatcaggaaacagctgaaaagaccaagagagtgagcaccaaagagatctaatgaggaggcacagaccagtgtctggatcccagcatcttctccacttcagcgctgagttcagtatacacaagtgtctgctacagtcgccaaatcaccagtatttgcttatatagcaatgagttttattttgtttatttgttttgcaataaaggatatgaaggtggctggctaggaagggaagggccacagccttcatttctaggagtgctttaagagaaactgtaaatggtgctctggggctggaggctagtaaggaaactgcatcacgattgaaagaggaacagacccaaatctgaacctcttttgagtttactgcatctgtcagcaggctgcagggagtgcacacgatgccagagagaacttagcagggtgtccccggaggagaggtttgggaagctccacggagaggaacgctctctgcttccagcctctttccattgccgtcagcatgacagacctccagcatccacgcatctcttggtcccaataactgcctctagatacatagccatactgctagttaacccagtgtccctcagacttggatggagtttctgggagggtacacccaaatgatgcagatacttgtatactttgagccccttagcgacctaaccaaattttaaaaatactttttaccaaaggtgctatttctctgtaaaacacttttttttggcaagttgactttattcttcaattattatcattatattattgttttttaatattttattttcttgactaggtattaagcttttgtaattatttttcagtagtcccaccacttcataggtggaaggagtttggggttcttcctggtgcaggggctgaaataacccagatgcccccaccctgccacatactagatgcagcccatagttggcccccctagcttccagcagtccactatctgccagaggagcaagggtgccttagaccgaagccaggggaagaagcatcttcataaaaaactttcaagatccaaacattaatttgtttttatttattctgagaagttgaggcaaatcagtattcccaaggatggcgacaagggcagccaagcagggcttaggatatcccagcctaccaatatgctcattcgactaactaggagggtgagttggccctgtctcttcttttttctggacctcagtttcctcagtgagctggtaagaatgcactaaccttttgatttgataagttataaattctgtggttctgatcattggtccagaggggagataggttcctgtgatttttccttcttctctatagaataaatgaaatcttgttactagaacaagaaatgtcagatggccaaaaacaagatgaccagatttgatctcagcctgatgaccctacaggtcgtgctatgatatggagtcctcatgggtaaagcaggaagagagtgggaaagagaaccaccccactctgtcttcatatttgcatttcatgtttaacctccggctggaaatagaaagcattcccttagagatgaggataaaagaaagtttcagattcaacagggggaagaaaatggagatttaatcctaaaactgtgacttggggaggtcagtcatttacagttagtcctgtgtctttcgacttctgtgattattaaccccactcactaccctgtttcagatgcatttggaataccaaagattaaatccttgacataagatctcatttgcagaaagcagattaaagaccatcagaaggaaattatttaggttgtaatgcacaggcaactgtgagaaactgttgtgccaaaaatagaattccttctagtttttcttgttctcatttgaaaggagaaaattccactttgtttagcatttcaagcttttatgtatccatcccatctaaaaactcttcaaactccacttgttcagtctgaaatgcagctccctgtccaagtgccttggagaactcacagcagcacgccttaatcaaaggttttaccagcccttggacactatgggaggagggcaagagtacaccaatttgttaaaagcaagaaaccacagtgtctcttcactagtcatttagaacatggttatcatccaagactactctaccctgcaacattgaactcccaagagcaaatccacattcctcttgagttctgcagcttctgtgtaaatagggcagctgtcgtctatgccgtagaatcacatgatctgaggaccattcatggaagctgctaaatagcctagtctggggagtcttccataaagttttgcatggagcaaacaaacaggattaaactaggtttggttccttcagccctctaaaagcatagggcttagcctgcaggcttccttgggctttctctgtgtgtgtagttttgtaaacactatagcatctgttaagatccagtgtccatggaaacattcccacatgccgtgactctggactatatcagtttttggaaagcagggttcctctgcctgctaacaagcccacgtggaccagtctgaatgtctttcctttacacctatgtttttaagtagtcaaacttcaagaaacaatctaaacaagtttctgttgcatatgtgtttgtgaacttgtatttgtatttagtaggcttctatattgcatttaacttgtttttgtaactcctgattcttccttttcggatactattgatgaataaagaaattaaagtgatggttttggtttcctttcccccaattaaggccaaataaagtcgtgagaacattacccattta